(19) United States
(12) Patent Application Publication
(10) Pub. No.: US 2021/0059578 A1
(43) Pub. Date: Mar. 4, 2021

(54) REAGENT COATED LENSES

(71) Applicant: INSCOPIX, INC., Palo Alto, CA (US)

(72) Inventors: Jonathan J. NASSI, Sunnyvale, CA (US); David CHENG, Palo Alto, CA (US); Mark TRULSON, San Jose, CA (US)

(21) Appl. No.: 17/020,978

(22) Filed: Sep. 15, 2020

Related U.S. Application Data
(63) Continuation of application No. PCT/US2019/023902, filed on Mar. 25, 2019.
(60) Provisional application No. 62/647,377, filed on Mar. 23, 2018.

(51) Int. Cl. A61B 5/1459; A61B 5/1473; A61B 5/145; A61B 5/00; A61K 9/00; A61K 49/00; A61K 35/761; C12N 7/00; C07K 14/435; C07K 14/47; C12N 9/12

(52) U.S. Cl. CPC A61B 5/1459; A61B 5/1473; A61B 5/14546; A61B 5/0042; A61K 9/0085; A61K 49/005; A61N 5/0601; C12N 7/00; C07K 14/43595; C07K 14/4728; C12N 9/12; C12Y 207/11018; A61K 49/0047; A61K 35/761

(57) ABSTRACT

Disclosed herein are implantable devices coated with a reagent, and methods of preparing and utilizing such devices. The implantable device may comprise an implantable light conduit. The present disclosure enables a more precise registration between the reagent and the device implanted in the target tissue of a subject.

### CROSS-REFERENCE

This application claims the benefit of U.S. Provisional Application No. 62/647,377, filed Mar. 23, 2018, which application is entirely incorporated herein by reference for all purposes.

### BACKGROUND

Various technologies for research, diagnostics, and therapeutics require delivery of reagents combined with implantation of a device to a targeted region in a tissue of a subject. This may require co-registration of the delivered reagent and the implanted device. This can be accomplished by multiple procedures, one procedure to deliver the reagent and a separate procedure to implant the device. Each procedure may pose significant risks to the health of the subject, especially when performed in separate surgeries. In addition, it may be very challenging to re-target the reagent delivery site to implant the device so that the device is in proper registration with the reagent. Therefore, there is a need for precise registration between the reagent and the device implanted in the brain or other tissues for research, diagnostics, and therapeutics.

### SUMMARY

The present disclosure relates to light conduit coated with a reagent. In various aspects, the present disclosure provides an implantable light conduit coated with a reagent, and methods of preparing and utilizing such devices.

Various technologies for research, diagnostics, and therapeutics for the central nervous system (CNS) rely on delivery of reagents (e.g. drugs, viruses, etc.) to a subject. In some instances, the delivery of reagents may be combined with implantation of a device to a targeted region of the brain of the subject. This may require co-registration of the delivered reagent and the implanted device. This can be accomplished by multiple procedures, and potentially in multiple surgeries, where one procedure delivers the reagent and a separate procedure implants the device. Each procedure may pose significant risks to the health of the subject, especially when performed as multiple penetrations or in multiple surgeries. In some instances, it may be very challenging to re-target the reagent delivery site to implant the device so that the device is in proper registration with the reagent. These challenges may reduce the overall success rate of the implant. Even when both procedures are combined within the same surgery, reagent delivery may be performed separately and independently from device implantation, thus requiring multiple (at least two) penetrations into the brain or other tissues. Multiple penetrations to the brain or target tissues may cause damage to the brain or the target tissues and/or may limit the viability of virus infection and transgene expression. In some instances, the reagent may pose a risk to the health of the subject, and there may be an interest to limit the volume of brain or other target tissues exposed to the reagent to the space immediately surrounding the device.

Amongst other things, the present disclosure recognizes and addresses these challenges, and may enable a more precise registration between the reagent and the device implanted in the brain or other target tissues. The present disclosure may eliminate the need for multiple surgeries and the challenges associated with re-targeting and may reduce the potential for brain damage or damage to the target tissue and unnecessary exposure to the reagent. The present disclosure may minimize the potential for error in alignment between the reagent and the implant. The potential for failure may come from lack of accurate targeting of the light conduit to the intended brain or target tissue region, but not from misalignment of the reagent and the implant. In some instances where the reagent is a virus or other expression systems, the success rate of expression at the site of the implantation of the light conduit can be high. In turn, the present disclosure may increase the overall success rate of the implant and the prognosis or outcome for the subject.

Provided herein are reagent coated light conduits (e.g., lenses), devices and systems comprising or using the same, methods comprising or using the same, and methods of storing the same. The reagent coated light conduits may be implantable. The reagent coated light conduits may be used with respect to subjects, such as live subjects. Beneficially, such use of reagent coated light conduits may obviate the need to have two separate operations (e.g., surgeries) to (a) implant the light conduit, and (b) deliver the reagent to the subject. A single operation of implanting the reagent coated light conduit may achieve both the implantation of the light conduit and the delivery of the reagents. In certain aspects, the reagent coated light conduit may be integrated with or otherwise affixed to a mechanical support structure, such as a baseplate. Beneficially, such integrated reagent coated light conduit-baseplate assembly may obviate the need to have three separate operations (e.g., surgeries) to (a) implant the light conduit, (b) deliver the reagent, and (c) install the baseplate (e.g., on the subject). A single operation of implanting the integrated reagent coated light conduit-baseplate assembly may achieve all of the implantation of the light conduit, the delivery of the reagent, and the installation of the baseplate. Such reduction in operations can significantly increase the success rate of preparing the subject for imaging. For example, a separate installation of the baseplate on the subject can require its precise securement to the subject's skull, which can be difficult given the use of adhesives that shrink during curing, which can then change their precise location. The separate baseplate installation procedure can also require that the subject be sedated, which typically renders neurons inactive, which in turn makes it difficult to determine the precise location to secure the baseplate (which can be guided by optimizing focus on neurons displaying active calcium dynamics).

In a certain aspect, described herein, is an implantable light conduit comprising a distal end comprising a surface configured to be implanted in a subject; a proximal end; and an elongate body extending between the distal end and the proximal end, where the implantable light conduit comprises one or more reagents configured to be delivered to the subject. In some aspects, the elongate body comprises a cylindrical shape. In some aspects, the elongate body comprises a square prism at the distal end. In some aspects, the light conduit comprises a lens. In some aspects, the lens comprises a gradient-index (GRIN) lens. In some aspects, the implantable light conduit is integrated with a mechanical element such as a baseplate. The baseplate may be attached to the proximal end or the elongate body, wherein the proximal end or elongate body is attached to a first surface of the baseplate configured to interact with the subject. The baseplate may be permanently attached to the proximal end or the elongate body. The baseplate may be detachably attached to the proximal end or the elongate body. The baseplate may comprise an interface configured to attach to a microscope. In some aspects, the light conduit comprises optical fibers. In some aspects, the light conduit comprises a glass window. In some aspects, the light conduit comprises a transparent tube comprising a lumen. In some aspects, the transparent tube comprises silica, fused quartz, or polymers. In some aspects, the light conduit comprises a volume equal to or less than 10 cm³. In some aspects, the light conduit comprises a volume equal to or less than 1 cm³. In some aspects, the light conduit comprises an electrode. In some aspects, the surface is substantially flat. In some aspects, the surface is curved. In some aspects, the surface comprises pores to which the one or more reagents adhere to. In some aspects, the pores are generated via etching the surface. In some aspects, the surface comprises the one or more reagents. In some aspects, the one or more reagents comprise drugs, viruses, cells, or other biological materials. In some aspects, the one or more reagents comprise adeno-associated viruses (AAV). In some aspects, the AAV encodes fluorescent calcium indicators. In some aspects, the AAV encodes opsins. In some aspects, the AAV encodes fluorescent voltage indicators. In some aspects, the AAV encodes static fluorescent reporters. In some aspects, the AAV encodes transgenes. In some aspects, the transgenes are configured to reduce inflammatory response, immune response, or provide therapeutic benefit at a site of implant. In some aspects, a concentration of the AAV deposited on the surface of the light conduit is between 10¹⁰ to 10¹⁶ viral genome/mL. In some aspects, the one or more reagents comprise secondary reagents. In some aspects, the secondary reagents comprise synthetic polymers or oligomers. In some aspects, the secondary reagents comprise naturally derived polymers or oligomers. In some aspects, the secondary reagents comprise polymeric conjugates, micelles, hydrogels, microparticles, nanoparticles, microspheres, or nanospheres. In some aspects, the secondary reagents are photodegradable. In some aspects, the secondary reagents are biodegradable. In some aspects, the secondary reagents are biodegradable by enzymatic or non-enzymatic hydrolysis. In some aspects, the secondary reagents are degradable by electrochemical methods. In some aspects, the secondary reagents are configured to protect drugs, viruses, cells, or other biological materials. In some aspects, the secondary reagents are configured to slow desorption of drugs, viruses, cells, or other biological materials from the light conduit at a controllable rate. In some aspects, the one or more reagents evenly cover the surface in its entirety. In some aspects, the one or more reagents are dry. In some aspects, the one or more reagents comprise two or more different reagents. In some aspects, the one or more reagents are pattern deposited on the surface. In some aspects, the one or more reagents are deposited on the surface in multiple layers. In some aspects, the body comprises a height of at least 1 mm. In some aspects, the one or more reagents are deposited onto the light conduit via droplets (e.g., single droplets or an array of smaller droplets). In some aspects, the one or more reagents are deposited onto the light conduit by dipping the light conduit into a solution comprising the one or more reagents. In some aspects, the one or more reagents are deposited onto the light conduit by spraying the one or more reagents onto the light conduit. In other aspects, described herein, is a method of storing the implantable light conduit comprising: storing the implantable light conduit in a controlled environment. In some aspects, the controlled environment comprises a temperature between −100 and 40 degrees Celsius. In some aspects, the controlled environment comprises a relative humidity between 0% and 50%. In some aspects, the controlled environment is protected from light. In some aspects, the controlled environment protects the light conduit from mechanical damage. In some aspects, the controlled environment is aseptic or sterile. In some aspects, the implantable light conduit is integrated with a mechanical element such as a baseplate.

In other aspects, described herein, is a method of directing light to a target site comprising: implanting the light conduit as described herein in the subject; contacting the target site of the subject with the surface of the implantable light conduit; transferring the one or more reagents of the light conduit to the target site; and directing light to the target site using the light conduit. In some aspects, the light conduit is integrated with a mechanical element such as a baseplate wherein the baseplate is attached to a subject.

In other aspects, described herein, is a light delivery system comprising a light source configured to generate light; the implantable light conduit as described herein, configured to direct the light to a target site in the subject to elicit a response; and a detector configured to receive the response. In some aspects, the implantable light conduit is integrated with a mechanical element such as a baseplate to interface with a subject comprising the target site. The baseplate may be permanently attached to the implantable light conduit. The baseplate may be detachably attached to the implantable light conduit. The baseplate may comprise an interface configured to attach to a microscope. In some aspects, the system comprises an imaging system. In some aspects, the system comprises an optogenetic stimulation system. In some aspects, the system is coupled together via a housing. In some aspects, the system comprises a combined volume equal to or less than 5 cm³. In some aspects, the system comprises a combined weight equal to or less than 100 gr. In some aspects, the system is a wireless system. In some aspects, the system is a wired system.

In other aspects, described herein, is a method of coating an implantable light conduit comprising a) providing the implantable light conduit, wherein the implantable light conduit comprises a distal end comprising a surface, a proximal end, and an elongate body extending there between; b) depositing a droplet or an array of droplets of one or more reagents onto the surface; and c) drying the droplet on the surface. In some aspects, the droplet comprises a volume between a range of about 1 to 1000 nL. In some aspects, the method further comprises repeating b) and c) one or more times. In some aspects, the method further comprises storing the implantable light conduit in a controlled environment. In some aspects, the controlled environment comprises a temperature between −100 and 40 degrees Celsius. In some aspects, the controlled environment comprises a relative humidity between 0% and 50%. In some aspects, the controlled environment is protected from light. In some aspects, the controlled environment protects the light conduit from mechanical damage. In some aspects, the controlled environment is aseptic or sterile. In some aspects, the said method is performed under aseptic or sterile conditions. In some aspects, the said method is performed at a predetermined temperature. In some aspects, step b) comprises patterned depositing of the one or more reagents. In some aspects, step b) comprises depositing a droplet or an array of droplets over the surface in its entirety. In some aspects, the one or more reagents comprise drugs, viruses, cells, or other biological materials. In some aspects, the one or more reagents comprise AAV. In some aspects, the AAV encodes fluorescent calcium indicators. In some aspects, the AAV encodes opsins. In some aspects, the AAV encodes fluorescent voltage indicators. In some aspects, the AAV encodes static fluorescent reporters. In some aspects, the AAV encodes transgenes. In some aspects, the transgenes are configured to reduce inflammatory response, immune response, or provide therapeutic benefit at a site of implant. In some aspects, a concentration of the AAV deposited on the surface of the light conduit is between 10¹⁰ to 10¹⁶ viral genome/mL. In some aspects, the one or more reagents comprise secondary reagents. In some aspects, the secondary reagents comprise synthetic polymers. In some aspects, the secondary reagents comprise naturally derived polymers. In some aspects, the secondary reagents comprise polymeric conjugates, micelles, hydrogels, microparticles, nanoparticles, microspheres, or nanospheres. In some aspects, the secondary reagents are degradable. In some aspects, the secondary reagents are photodegradable. In some aspects, the secondary reagents are biodegradable. In some aspects, the secondary reagents are degradable by electrochemical methods. In some aspects, the secondary reagents are configured to protect drugs, viruses, cells, or other biological materials. In some aspects, the secondary reagents are configured to desorb drugs, viruses, cells, or other biological materials at a controllable rate. In some aspects, the implantable light conduit is integrated with a mechanical element such as a baseplate.

In other aspects, described herein, is a method for directing light to a target site of a subject, the method comprising bringing into contact a light conduit with the subject, wherein the light conduit comprises one or more reagents; releasing the one or more reagents from the light conduit; delivering the one or more reagents to the target site of the subject, wherein delivery of the one or more reagents is substantially restricted to an area corresponding to where the one or more reagents had been located on the light conduit prior to said releasing; and with aid of the light conduit, directing light to the target site of the subject. In some aspects, the light conduit is integrated with a mechanical element such as a baseplate. In some aspects, the method further comprises imaging the target site of the subject. In some aspects, the method further comprises stimulating the target site of the subject via said light. In some aspects, bringing into contact comprises implanting the light conduit in the subject. In some aspects, imaging the target site comprises directing light to the target site via the light conduit, inducing a response from the target site, and directing the response with aid of the light conduit to a detector. In some aspects, the target site comprises brain tissue. In some aspects, the light conduit comprises a cylindrical structure. In some aspects, implanting the light conduit and said delivering the one or more reagents is accomplished via a single penetration into the subject. In some aspects, implanting the light conduit and said delivering the one or more reagents is accomplished in a single surgery. In some aspects, a success rate of said obtaining co-registration between the one or more reagents in the target site and the light conduit is equal to or more than 90%. In some aspects, the one or more reagents are located on a surface of the light conduit. In some aspects, the target site comprises a surface area substantially equal to the light conduit's surface area. In some aspects, the one or more reagents comprise drugs, viruses, cells, or other biological materials. In some aspects, the one or more reagents comprise AAV. In some aspects, the AAV encodes fluorescent calcium indicators. In some aspects, the AAV encodes opsins. In some aspects, the AAV encodes fluorescent voltage indicators. In some aspects, the AAV encodes static fluorescent reporters. In some aspects, the AAV encodes transgenes. In some aspects, the transgenes are configured to reduce inflammatory response, immune response, or provide therapeutic benefit at a site of implant. In some aspects, a concentration of the AAV deposited on the surface of the light conduit is between 10¹⁰ to 10¹⁶ viral genome/mL. In some aspects, the one or more reagents comprise secondary reagents. In some aspects, the secondary reagents comprise synthetic polymers. In some aspects, the secondary reagents comprise naturally derived polymers. In some aspects, the secondary reagents comprise polymeric conjugates, micelles, hydrogels, microparticles, nanoparticles, microspheres, or nanospheres. In some aspects, the one or more reagents are degradable. In some aspects, the one or more reagents are photodegradable. In some aspects, the secondary reagents are biodegradable. In some aspects, the secondary reagents are degradable by electrochemical methods. In some aspects, the secondary reagents are configured to protect drugs, viruses, cells, or other biological materials. In some aspects, the secondary reagents are configured to desorb drugs, viruses, cells, or other biological materials at a controllable rate. In some aspects, releasing occurs passively. In some aspects, releasing occurs with aid of light. In some aspects, releasing occurs with aid of electrochemical methods. In some aspects, the light conduit comprises one or more lenses. In some aspects, the light conduit comprises a glass window. In some aspects, the light conduit comprises optical fibers.

In other aspects, described herein, is a baseplate configured to be mounted on a live being. The baseplate comprises a first surface configured to interface with the live being and a second surface opposite the first surface, wherein the second surface is configured to interface with a microscope. A light conduit may be integrated with the first surface, wherein the light conduit is at least partially implantable in the live being and comprises one or more reagents configured to be delivered to the live being.

In other aspects, described herein, is a method for imaging a target region in a subject, the method comprising mounting an integrated baseplate on a subject, mounting a microscope on the baseplate, and activating the microscope. The method includes providing an integrated baseplate assembly configured to mount o the subject having the target region, wherein the integrated baseplate comprises an interface configured to detachably attach a microscope configured to image the target region, wherein the baseplate is integrated with a light conduit. The light conduit may be partially implanted in the subject. Concurrent with mounting the microscope on the baseplate, the microscope may be aligned. The microscope may be detached from the baseplate with one hand of an operator. The baseplate may comprise a first surface configured to interface with the subject, wherein the light conduit is integrated with the first surface, wherein the light conduit comprises one or more reagents configured to be delivered to the subject. The baseplate may comprise a second surface opposite the first surface, wherein the second surface is configured to interface with the microscope. The method may further comprise mechanical elements wherein repeated removal and attachment of the microscope to the second surface provides a consistent field of view of within 50 micrometers lateral and a focus depth of 25 micrometers relative to a distal end of the second surface.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1A shows examples of light conduits, including at least the cylinder, a glass coverslip, and a glass tube comprising a lumen into which the GRIN lens can be placed, in accordance with some embodiments.

FIG. 1B shows examples of baseplates integrated with a light conduit, in accordance with some embodiments.

FIG. 2 shows a flow chart of a method for directing light to a target site of a subject by a single procedure to place a light conduit comprising reagents to the target site and deliver the reagents to the target site, in accordance with some embodiments.

FIG. 3 shows a schematic diagram of a co-registration between the reagent delivery and the light conduit, in accordance with some embodiments.

FIG. 4 shows a schematic diagram of an exemplary microscope and components that may be coupled to the light conduit, in accordance with some embodiments.

FIG. 5 shows examples of methods of coating the light conduit, including a direct deposition by a single droplet, direction deposition by an array of droplets, dipping, and spraying, in accordance with some embodiments.

FIG. 6A shows a prism probe immediately following application of a droplet comprising virus that was pipetted onto imaging face of the lens, in accordance with some embodiments.

FIG. 6B shows a prism probe at a first time point following application of a droplet comprising virus that was pipetted onto imaging face of the lens, in accordance with some embodiments.

FIG. 6C shows a prism probe at a second time point following application of a droplet comprising virus that was pipetted onto imaging face of the lens, in accordance with some embodiments.

FIG. 7A shows a straight probe immediately following application of a droplet comprising virus that was pipetted onto imaging face of the lens, in accordance with some embodiments.

FIG. 7B shows a straight probe at a first time point following application of a droplet comprising virus that was pipetted onto imaging face of the lens, in accordance with some embodiments.

FIG. 7C shows a straight probe at a second time point following application of a droplet comprising virus that was pipetted onto imaging face of the lens, in accordance with some embodiments.

FIG. 7D shows a straight probe at a third time point following application of a droplet comprising virus that was pipetted onto imaging face of the lens, in accordance with some embodiments.

FIG. 8A shows a still image from a video of in vivo calcium imaging following virus-coated prism probe lens implantation, in accordance with some embodiments.

FIG. 8B shows a still image from a video of in vivo calcium imaging following virus-coated straight probe lens implantation, in accordance with some embodiments.

FIGS. 9A-C show post-mortem histology showing extent of viral infection and GCaMP6 expression surrounding the implanted lens from the same case as shown in FIG. 8A, in accordance with some embodiments.

FIGS. 10A-B show the storage of virus-coated prism probe lenses stored inside a plastic petri dish at room temperature at low magnification (FIG. 10A) and higher magnification (FIG. 10B), in accordance with some embodiments.

### DETAILED DESCRIPTION

The present disclosure generally relates to implantable devices and methods. More specifically, the present disclosure relates to implantable devices coated with a reagent. The implantable device may include, but is not limited to, an implantable light conduit. A light conduit as described herein may refer to any component or apparatus that is capable of passing or transmitting light. For example, a light conduit may include, but is not limited to, gradient-index (GRIN) lenses, optical fibers, and glass cover slips, and related devices. As described herein, there may be challenges associated with co-registration of the delivered reagent and the implanted device when the reagent and the device are delivered in multiple procedures. The present disclosure may enable a more precise registration between the reagent and the device implanted in the brain or other target tissues. The present disclosure may help eliminate the need for multiple surgeries and the challenges associated with re-targeting, and may reduce the potential for brain or target tissue damage and unnecessary exposure to the reagent. In turn, the present disclosure may increase the overall success rate of the implant and the prognosis or outcome for the subject.

Beneficially, such use of reagent coated light conduits may obviate the need to have two separate operations (e.g., surgeries) to (a) implant the light conduit, and (b) deliver the reagent to the subject. A single operation of implanting the reagent coated light conduit may achieve both the implantation of the light conduit and the delivery of the reagents.

In certain aspects, the reagent coated light conduit may be integrated with or otherwise affixed to a mechanical support structure, such as a baseplate. Beneficially, such integrated reagent coated light conduit-baseplate assembly may obviate the need to have three separate operations (e.g., surgeries) to (a) implant the light conduit, (b) deliver the reagent, and (c) install the baseplate (e.g., on the subject). By reducing the number of surgeries, the animal is only subjected to sedation a single time. Sedation typically renders neurons inactive which in turn interferes with the ability to use neurons displaying active calcium dynamics as guides in the placement of the baseplate and microscope. Additionally, the integrated baseplate does not require an intermediate placement step where the light conduit is secured by adhesives. Adhesives may shrink after application, affecting the precision of the placement. An integrated baseplate may save time and increase the accuracy of the procedure. A single operation of implanting the integrated reagent coated light conduit-baseplate assembly may achieve all of the implantation of the light conduit, the delivery of the reagent, and the installation of the baseplate.

Described in the present disclosure may be a device that can be implanted in a subject. Optionally, the device may be chronically implanted. The device may comprise a light conduit. The device may comprise or be an endoscope. The device may be used for various purposes. For example, the device may be implanted in the brain of a subject and deliver reagent to the brain tissue surrounding it. For example, the device may be implanted in a target tissue of a subject and deliver a reagent to the target tissue surrounding it. In addition or alternatively, the device may be used to monitor or affect the subject. For example, the device may be used to sense, including but not limited to electrical signals, chemical signals, optical signals, or combinations thereof, of the subject. The signals may be recorded and analyzed by the device. In some instances, the device may be used to manipulate the activity of neuronal and/or other cell types, physiological targets within CNS, targets associated with the cardiovascular system, targets associated with the immune system, or combinations thereof. For example, the device may manipulate by electrical stimulation, chemical stimulation, optical stimulation, optogenetic stimulation, or combinations thereof. The physiological targets may be a tumor or a malignancy, and the application of the device may affect the growth of the tumor. In some instances, the device may be used to treat a condition in the subject. For example, the treatment may be by delivery of a therapeutic agent from the light conduit coated with the therapeutic agent.

In some instances, an endoscope comprising a reagent-coated light conduit is affixed to a mechanical element (e.g., coupler, mounting plate, baseplate, etc.) that permits an integrated microscope to be accurately mounted to a subject. Such integrated assembly may permit the microscope to be mounted to a subject in a fixed position relative to the endoscope yet be readily removable and re-mountable with high precision. The mechanical precision of the mating of the endoscope and the microscope may be sufficient to provide consistent field of view and/or consistent focus depth relative to a distal end of the endoscope, even after repeated removal and remounting of the integrated microscope. In some instances, for example, a consistent field of view (lateral) of within about 100 micrometers (μm), 90 μm, 80 μm, 70 μm, 60 μm, 50 μm, 40 μm, 30 μm, 20 μm, 10 μm, 5 μm, or less may be achieved. Alternatively or in addition, a consistent focus depth of within about 50 μm, 45 μm, 40 μm, 35 μm, 30 μm, 25 μm, 20 μm, 15 μm, 10 μm, 5 μm or less, relative to the distal end of the endoscope, may be achieved.

One aspect of the present disclosure provides for a baseplate configured to be mounted to a live being. The baseplate can comprise a first surface for interfacing with the live being, and a second surface opposite the first surface for interfacing with a microscope or other imaging system. In some instances, the baseplate can comprise an interface configured to couple with an external instrument, wherein the external instrument is configured to aid in an experiment involving the microscope. In some instances, the interface may be on the first surface for interfacing with the live being. Alternatively or in addition, the interface may be on the second surface for interfacing with the microscope or other imaging system. In some instances, the external instrument may be detachably coupled to the interface. In some instances, the external instrument may be permanently affixed to the interface. In some instances, the external instrument may comprise an optical element. The external instrument may comprise a light conduit. In one aspect, a light conduit is integrated with the baseplate. The light conduit may be coated with a reagent, as described elsewhere herein. In some embodiments, the baseplate can comprise a size of at least about 20%, 25%, 30%, 35%, 40%, 45%, 50%, 55% , or greater of a live being's skull area, thereby improving adhesion between the baseplate and live being. Alternatively or in addition, the baseplate can comprise a size of at most about 55%, 50%, 45%, 40%, 35%, 30%, 25%, 20%, or less, of a size of a live being's skull, such as to minimize disturbance to the live being when the baseplate is mounted to its skull. In some embodiments, the baseplate can comprise a maximum dimension of at most about 2.5 inches, 2 inches, 1.5 inches, 1 inch, 0.5 inches, or less. Alternatively or in addition, the baseplate can comprise a maximum dimension of at least about 0.5 inches, 1 inch, 1.5 inches, 2 inches, 2.5 inches, or greater. Optionally, the baseplate can comprise a plurality of openings each configured to be coupled to a light conduit. For example, the light conduit can be a GRIN lens. In another example, the second surface of the baseplate can be configured to be interchangeably attached to a variety of microscopes.

Mounting and dismounting of the microscope to the baseplate may be optimized for one-handed operation. For example, the microscope may be readily attached to the baseplate using one hand (e.g., of an operator). The microscope may be readily detached from the baseplate using one hand (e.g., of an operator). In some instances, the microscope may be readily attached or detached from the baseplate via a quick attachment/release mechanism (e.g., pressing a lever, pushing a button, pulling, applying pressure, etc.) that requires only one hand to operate (e.g., at most five digits) or trigger the attachment or detachment.

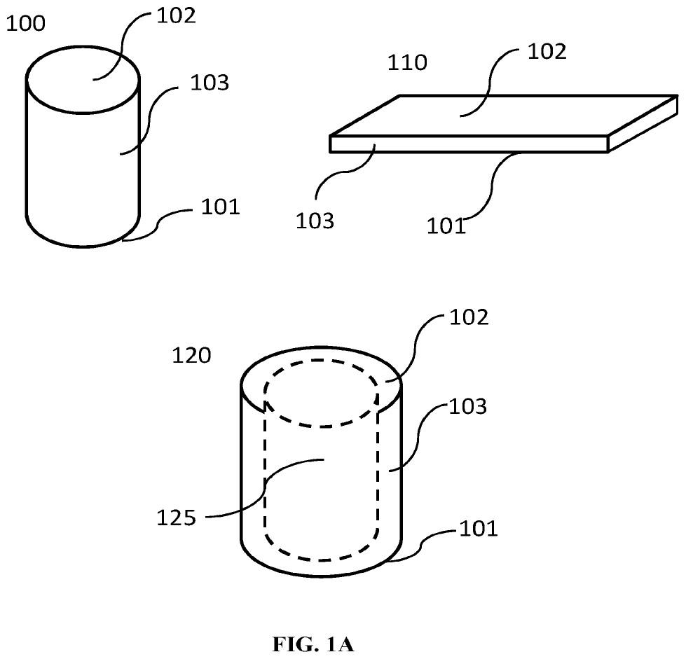

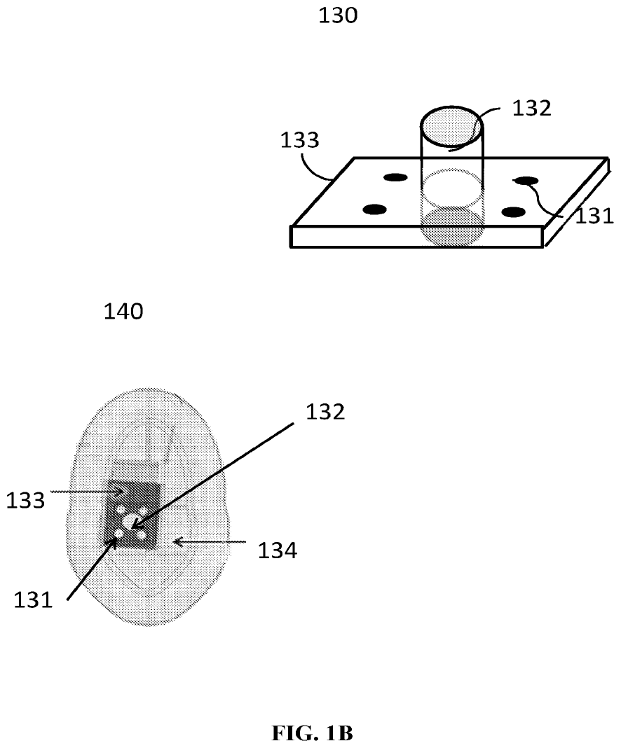

FIG. 1A illustrates various light conduits, in accordance with embodiments. As described herein, the light conduit may comprise a lens (e.g., such as a GRIN lens) 100, a glass window 110, or a transparent tube 120 comprising a lumen 125. The light conduit may be configured to be coupled with a subject. Optionally, the light conduit may be implantable. For example, the light conduit may be implanted within a subject (e.g., an animal) in various locations, such as in or near a brain of the subject. In one example, the lens 100 may be configured to be implanted in a brain of the subject. In another example, the glass window 110 (which may be a glass coverslip) may be configured to be placed onto a brain of a subject to be in direct contact with a target tissue of interest and provide a cranial window to the brain of the subject. In another example, the transparent tube 120 may be configured to be implanted in a brain of a subject. Subsequently, various other components such as GRIN lenses may be coupled with the transparent tube 120. As described above, the light conduit may be configured to, or may be capable of transmitting light.

The light conduit, such as the GRIN lens, may be integrated with a baseplate. Beneficially, such integrated light conduit-baseplate assembly may obviate the need to have two separate operations (e.g., surgeries) to (a) implant the light conduit and (b) install the baseplate (e.g., on the subject). FIG. 1B illustrates an integrated light conduit-baseplate assembly. An integrated baseplate 130 can comprise a light conduit 132 and a baseplate 133. The baseplate 133 may comprise a first surface configured to interface with a subject 140, and a second surface configured to interface with a microscope (or other imaging system or other instrument). The first surface may be opposite the first surface. The light conduit 132 may be integrated with the baseplate 133 on the first surface configured to interface with the subject 140. Alternatively or in addition, the light conduit may be integrated with the baseplate on the second surface. Alternatively or in addition, the light conduit may be integrated with the baseplate on both the first surface and the second surface. As described elsewhere herein, the baseplate 133 may be integrated with a GRIN lens or other light conduit. As described elsewhere herein, one or more surfaces of the light conduit may be coated with one or more reagents. The integrated baseplate 130 may be designed to attach to the skull 134 of the subject 140, such as via the first surface configured to interface with the subject. The integrated baseplate 130 may be designed to be attached to a microscope (or other imaging system or other instrument) through an interface 131. In some instances, the interface 131 may permanently couple the microscope to the integrated baseplate. In other instances, the interface 131 may allow detachable coupling of the microscope to the integrated baseplate, and allow the microscope to be removed (once or multiple times). While FIG. 1B illustrates one embodiment of the interface 131 (e.g., thread holes) to couple to a microscope (or other structure), the interface may take any form, shape, or size that facilitates coupling of a microscope (or other imaging system or instrument) to the integrated baseplate 130. The interface may comprise any one or more fastening mechanism described herein.

In some instances, the light conduit 132 and the baseplate 133 may be directly fastened together as an integrated assembly. Alternatively, the light conduit and the baseplate may be indirectly fastened together as an integrated assembly, such as by coupling of an intermediary mechanical structure. In some instances, the baseplate may comprise an interface for fastening to the light conduit or an intermediary mechanical structure. The two components may be fastened together by any fastening mechanism, or a combination thereof. Examples of fastening mechanisms may include, but are not limited to, complementary threading, form-fitting pairs, buttons, nuts and bolts, snap-ons, hooks and loops, latches, threads, screws, staples, clips, clamps, prongs, rings, brads, rubber bands, rivets, grommets, pins, ties, snaps, Velcro, adhesives (e.g., glue), tapes, vacuum, seals, magnets, magnetic seals, a combination thereof, or any other types of fastening mechanisms.

The fastening can be temporary, such as to allow for subsequent unfastening of the two components without damage (e.g., permanent deformation, disfigurement, etc.) to the two components or with minimal damage. The fastening can be permanent, such as to allow for subsequent unfastening of the two components only by damaging at least one of the two components. One of the two components, or both, can be temporarily or permanently deformed (e.g., stretched, compressed, etc.) and/or disfigured (e.g., bent, wrinkled, folded, creased, etc.) or otherwise manipulated when fastened to each other or during fastening. In some instances, one or both of the two components can be cut into or pierced by the other when the two components are fastened together.

In some instances, the light conduit 132 may be fastened to the baseplate 133 after coating of a surface of the light conduit with one or more reagents. In some instances, the light conduit may be fastened to the baseplate prior to coating of a surface of the light conduit with one or more reagents. In some instances, the light conduit may be fastened to the baseplate after implantation into a subject. In some instances, the light conduit may be fastened to the baseplate prior to implantation into a subject. For example, an integrated light conduit-baseplate assembly may be implanted into the subject, such that the light conduit is at least partially implanted and the baseplate is affixed to the subject. In another example, a light conduit may be implanted into the subject, and then in a subsequent operation, the baseplate may be mounted onto the subject.

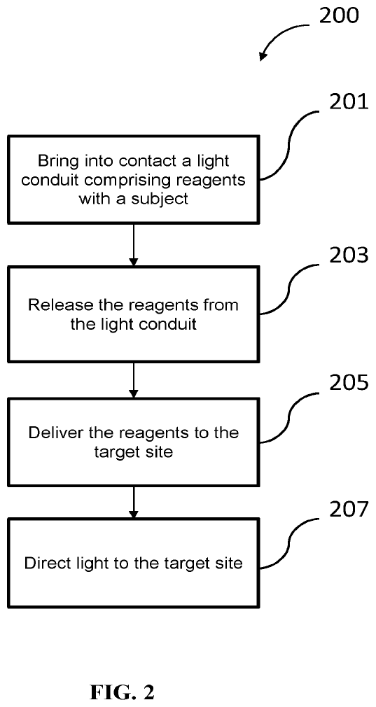

A reagent-coated light conduit may be delivered to the subject in a single procedure. FIG. 2 shows a flow chart of a method 200 for delivering a reagent-coated light conduit to a target site in a subject. In an operation 201, a light conduit comprising one or more reagents may be brought into contact with a target site in a subject. The reagent may be drugs, viruses, cells, or biological materials, such as adeno-associated viruses (AAV). The reagents may provide a desired response, for example, a reduction in an inflammatory response in the subject, or provide a therapeutic effect in the subject. In some instances, the reagents may aid fluorescent imaging of the target site. The target site may be a region in the brain. The target site may be another tissue. In an operation 203, the reagents may be released from the light conduit to the target site in contact with the light conduit. In some instances, operation 203 may happen without an external stimuli, e.g., automatically. Optionally, operation 203 may happen with aid of an external stimuli, e.g., a change in temperature, optical stimuli, thermal stimuli, electrical stimuli, etc. In an operation 205, the reagents may be delivered to the target site and the cells in the target site. For example, cell activity indicators or light sensitive polypeptides encoded by AAV in the reagents may transfect the cells at the target site. In an operation 207, light may be directed to the target site by the light conduit. For example, the delivered light may trigger an optical response by the cell activity indicators (such as fluorescent calcium or voltage indicators) in the transfected cells at the target site, which can be captured by the light conduit. A further operation may comprise mounting a microscope baseplate on the subject. Alternatively or in addition, the light conduit and the microscope baseplate may be integrated or otherwise fastened prior to contact with the subject (e.g., implantation), and the light conduit-microscope baseplate assembly may be brought into contact with the subject such as to contact the light conduit to the target site, and affixing (e.g., mounting) the baseplate onto the subject. In other examples, the light sensitive polypeptides may respond to the delivered light and control the activity of the transfected cells at the target site. The operations described here may be completed in a different order to deliver a light conduit and reagents in a single procedure. Operations may be added or deleted. FIG. 3 shows a schematic diagram of the co-registration between delivered reagents 310 and the light conduit 300 at the target site. The reagents may be released into a region close to or matching the surface of the light conduit.

The light conduit may be coupled to a light delivery system. In some instances, the light conduit may be coupled to a baseplate, which baseplate is coupled to the light delivery system, as described elsewhere herein. In some embodiments, the light conduit and the baseplate may be an integrated assembly. In other embodiments, the light conduit and the baseplate may be separate components. The light conduit and the light delivery system may be substantially permanently coupled or detachably coupled. The light conduit and the baseplate may be substantially permanently coupled or detachably coupled. The baseplate and the light delivery system may be substantially permanently coupled or detachably coupled.

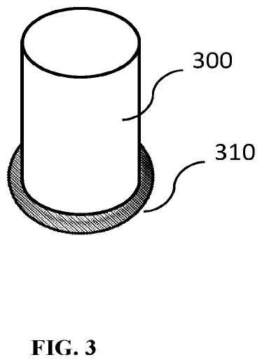

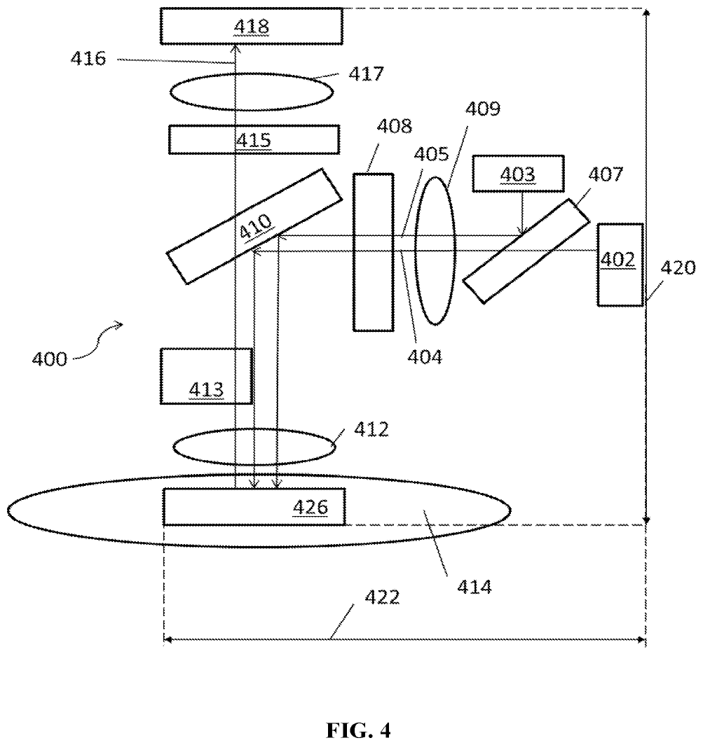

The light delivery system may be an imaging system or an optogenetic stimulation system or a combination thereof. In some instances, the imaging system may be a microscope as shown in FIG. 4. FIG. 4 shows a schematic diagram of an exemplary microscope and components that may be coupled to the light conduit. The system may deliver light to the target site through the light conduit. The delivered light may initiate a release of the reagents from the light conduit or an activity by the reagents. Other cues, such as electrical or chemical cues, may be delivered before, with, or after the delivered light to initiate a release of the reagents from the light conduit or an activity by the reagents. The system may sense and record various signals, such as optical, electrical, or chemical signals, from the target site for further analysis. The use of the reagent-coated light conduit may allow for precise co-registration of the reagent delivery and the light delivery. The use of the reagent-coated light conduit may allow for precise co-registration of the reagent delivery and sensing of signals, where the signals may be generated by the activity of the reagent.

The implantable light conduit may comprise a distal end comprising a surface configured to be implanted in a subject, a proximal end, and a body extending between the distal end and the proximal end. Optionally, the body may be an elongate body. For example, the lens 100 may have a distal end 101, a proximal end 102, and a body 103 as shown in FIG. 1. For a glass window 110 with a proximal end 102 and a body 103, a distal end 101 may be placed in direct contact with the target tissue. In another example, the transparent tube 120 comprising a lumen 125 as shown in FIG. 1 has a distal end 101, a proximal end 102, and a body 103. The light conduit may comprise one or more reagents configured to be delivered to the subject. The elongate body of the light conduit may comprise a cylindrical shape. Optionally, the body may comprise a rectangular shape, or any other shape (e.g., irregular, ellipsoidal, etc). In some instances, the light conduit may comprise a lens, a GRIN lens, optical fibers, a cover slip, a glass window, a transparent tube comprising a lumen, a prism, a probe, an optical probe, an electrophysiology probe, a neural probe, or combinations thereof. In some instances, the light conduit may provide a cranial window to a subject. In such instances, the light conduit may comprise a brain-implantable device. Optionally, the light conduit may comprise a periscope cannula. Alternatively or in addition, the light conduit may comprise one or more lenses.

The light conduit may comprise a prism probe. For example, the body (e.g., elongate body) may comprise a prism at the distal end, with an imaging surface. The imaging surface may be substantially flat, or may have a curvature. The prism may be a rectangular prism, a square prism, or may be a prism comprising cylindrical or irregular shapes.

The light conduit in some instances may comprise an electrode. The electrodes may optionally be utilized to provide a stimulus to a subject.

The light conduit may have imaging surfaces. The light conduit may have one or more imaging surfaces. The imaging surfaces may be on various surfaces of the light conduit. For example, the imaging surface may be at the distal end of the light conduit along the height of the body. Alternatively or in addition, and the imaging surface may be along the height of the body.

The imaging surface may be of any form. For example, the imaging surface may be substantially flat. Alternatively, the imaging surface may be curved. For example, the imaging surface may be concave or convex. In some instances, the imaging surface may be cylindrical. Optionally, the imaging surface may be rectangular or square.

The light conduit may be made of various materials. The light conduit may be made of materials that are transparent. In some instances, the light conduit may comprise glass, silica, fused silica, quartz, silicon, plastics, polymers, or combinations thereof.

The light conduit may comprise a volume equal to or less than 10 cm³. In some instances the light conduit may comprise a volume equal to or less than 0.1 cm³, 0.2 cm³, 0.3 cm³, 0.4 cm³, 0.5 cm³, 0.6 cm³, 0.7 cm³, 0.8 cm³, 0.9 cm³, 1.0 cm³, 1.1 cm³, 1.2 cm³, 1.3 cm³, 1.4 cm³, 1.5 cm³, 1.6 cm³, 1.7 cm³, 1.8 cm³, 1.9 cm³, or 2.0 cm³. In some instances the light conduit may comprise a volume equal to or less than 3 cm³, 4 cm³, 5 cm³, 6 cm³, 7 cm³, 8 cm³, 9 cm³, 10 cm³, 11 cm³, 12 cm³, 13 cm³, 14 cm³, 15 cm³, 16 cm³, 17 cm³, 18 cm³, 19 cm³, or 20 cm³. In some instances the light conduit may comprise a volume equal to or less than 20 cm³, 30 cm³, 40 cm³, 50 cm³, 60 cm³, 70 cm³, 80 cm³, 90 cm³, or 100 cm³.

In some instances the body of the light conduit may comprise a height that is equal to or less than about 0.1 mm, 0.2 mm, 0.3 mm, 0.4 mm, 0.5 mm, 0.6 mm, 0.7 mm. 0.8 mm, 0.9 mm, 1.0 mm, 1.5 mm, 2.0 mm, 2.5 mm, 3.0 mm, 3.5 mm, 4.0 mm, 4.5 mm, 5.0 mm, 6 mm, 7 mm, 8 mm, 9 mm, 10 mm, 12 mm, 14 mm, 16 mm, 18 mm, 20 mm, 22 mm, 24 mm, 26 mm, 28 mm, 30 mm, 35 mm, 40 mm, 45 mm, 50 mm, 55 mm, 60 mm, 65 mm, 70 mm, 75 mm, 80 mm, 85 mm, 90 mm, 95 mm, 100 mm, or any value therebetween. In some instances the body of the light conduit may comprise a height that is equal to or less than about 5 mm, 6 mm, 7 mm, 8 mm, 9 mm, 10 mm, 15 mm, 20 mm, 30 mm, 40 mm, 50 mm, 60 mm, 70 mm, 80 mm, 90 mm, 100 mm, or any value therebetween. In some instances the body of the light conduit may comprise a height that is equal to or less than about 10 cm, 15 cm, 20 cm, 25 cm, or 30 cm.

In some instances the body of the light conduit may comprise a height that is at least 0.5 mm, 1.0 mm, 1.5 mm, 2.0 mm, 2.5 mm, 3.0 mm, 3.5 mm, 4.0 mm, 4.5 mm, or 5.0 mm. In some instances the body of the light conduit may comprise a height that is at least 0.1 mm, 0.2 mm, 0.3 mm, 0.4 mm, 0.5 mm, 0.6 mm, 0.7 mm. 0.8 mm, 0.9 mm, 1.0 mm, 1.5 mm, 2.0 mm, 2.5 mm, 3.0 mm, 3.5 mm, 4.0 mm, 4.5 mm, or 5.0 mm. The body of the light conduit may comprise a height that is at least 5 mm, 6 mm, 7 mm, 8 mm, 9 mm, 10 mm, 15 mm, 20 mm, 30 mm, 40 mm, 50 mm, 60 mm, 70 mm, 80 mm, 90 mm, or 100 mm. In some instances the body of the light conduit may comprise a height that is at least 10 cm, 15 cm, 20 cm, 25 cm, or 30 cm.

The light conduit may comprise a cross-sectional distance in a plane that is perpendicular to the height of the body, where the cross-sectional distance is the longest distance in that plane. The cross-sectional distance may be a diameter for the light conduit with the plane that approximates a circle. In some instances the light conduit may comprise a cross-sectional distance that is equal to or less than 0.01 mm, 0.02 mm, 0.03 mm, 0.04 mm, 0.5 mm, 0.06 mm, 0.07 mm. 0.08 mm, 0.09 mm, 0.1 mm, 0.2 mm, 0.3 mm, 0.4 mm, 0.5 mm, 0.6 mm, 0.7 mm. 0.8 mm, 0.9 mm, 1.0 mm, 1.5 mm, 2.0 mm, 2.5 mm, 3.0 mm, 3.5 mm, 4.0 mm, 4.5 mm, or 5.0 mm. In some instances the light conduit may comprise a cross-sectional distance that is at least 0.01 mm, 0.02 mm, 0.03 mm, 0.04 mm, 0.5 mm, 0.06 mm, 0.07 mm. 0.08 mm, 0.09 mm, 0.1 mm, 0.2 mm, 0.3 mm, 0.4 mm, 0.5 mm, 0.6 mm, 0.7 mm. 0.8 mm, 0.9 mm, 1.0 mm, 1.5 mm, 2.0 mm, 2.5 mm, 3.0 mm, 3.5 mm, 4.0 mm, 4.5 mm, or 5.0 mm.

The light conduit may be placed in to the tissue of interest, or a target site. The target site may be the brain. In some instances the target site may be specific regions of the brain. For example, the target site may be the cortex, the striatum, the primary somatosensory cortex, and/or the dorsal striatum. The target site may be another tissue in a subject.

In some instances, the light conduit may be configured to be implanted into the target site. For example, the light conduit may be fully implanted into the target site. Alternatively, the light conduit may be partially implanted into the target site, where only a portion of the light conduit is placed into the target site. In some instances, the light conduit may not be implanted but may be coupled to the target site. For example, the light conduit may be laid on atop the target site. In such instances, the light conduit may or may not be in direct contact with the target site.

The light conduit may be capable of transmitting signals. The signal may be light, electrical, or chemical signals. The light conduit may be capable of transmitting a signal to and from the target site. The light conduit may transmit signals for imaging. The signal may modulate the activity of the reagent. The signal may modulate the activity of the cells and environment at the target site. The cells may respond to the signal by changes in fluorescent signal or other markers of cell activity. In some instances, once the reagent-coated light conduit is placed at the target site, a type of signals may be transmitted through the light conduit to the target tissue to modulate the activity of the delivered reagents, and another signal may be generated by the reagents and captured by the light conduit. The signal generated and captured may be of a different form than the signal transmitted to the target tissue. In some instances, light signals may be transmitted to the target tissue to modulate the activity of the delivered reagents. The reagents may comprise AAV viral vectors encoding fluorescent indicators of cell function, such as calcium indicators for neurons, that can transfect cells at the target site. The light signals transmitted to the target site may excite the fluorescent indicators expressed by the transfected cells at the target site. The emitted fluorescence signals from the fluorescent indicators may be captured by the light conduit and may provide an indication of the level of cell activity. In some instances, the reagents may comprise AAV viral vectors encoding light sensitive polypeptides, such as opsins, that can transfect cells at the target site and the light signals transmitted to the target site may modulate the activity of light sensitive polypeptides expressed by the transfected cells at the target site. The light sensitive polypeptides may generate light, electrical, chemical, or electrochemical signals in response to the light signal and cell activity, and the generated signals may be captured by the light conduit.

The distal end of the light conduit may comprise a non-imaging probe. For example, the distal end of the light conduit may comprise a probe for sensing electrical and/or chemical activity of the target site. The distal end of the probe may comprise an electrode. The distal end of the probe may comprise a tapered end, with a narrow tip that may be beneficial for specific, local sensing of electrical and/or chemical activity with a good spatial resolution.

In some instances, the light conduit may interface with a light delivery system. The system may be an imaging system, an optogenetic stimulation system, a system for neuromodulation, or a system for sensing. The system may perform more than one of these functions. As one example, an imaging system (e.g., together with the light conduit) can comprise a light source configured to generate light, a reagent-coated light conduit that is configured to direct the light to a target site in the subject to elicit a response, and a detector configured to receive the response. The imaging system can be coupled together via a housing. The imaging system may comprise a combined volume equal to or less than 10 cm³. The imaging system may comprise a combined volume equal to or less than 1 cm³. The imaging system may comprise a combined volume equal to or less than 0.1 cm³, 0.5 cm³, 1 cm³, 2 cm³, 3 cm³, 4 cm³, or 5 cm³. The imaging system may comprise a combined weight equal to or less than 100 grams. The imaging system may comprise a combined weight equal to or less than 50 g, 100 g, 200 g, 300 g, 400 g, or 500 g. The imaging system can be a wireless system. The signals may be transmitted by Bluetooth, Wi-Fi, LTE, or other wireless communication systems. The imaging system can be a wired system. The signals may be transmitted by cables in the wired system.

The imaging system that interfaces with the light conduit may be a microscope. FIG. 4 shows a schematic diagram of an exemplary microscope and components that may be coupled to the light conduit. The microscope system 400 can include a plurality of optical elements (e.g., lenses, filters, mirrors, dichroics, etc.) within the dimensions 420 and 422 for the imaging of a target object 414, such as the target site in the subject. The optical elements can include a first optical arrangement 402 (e.g., light sources, diodes, fiber optics) that can generate a first excitation light 404, a second optical arrangement 403 (e.g., light sources, diodes, fiber optics) that can generate a second excitation light 405, a light source combining element 407 (e.g., dichroic filter), a condenser lens 409, an excitation filter 408 (e.g., short pass filter, band pass filter), an objective lens 412, (dichroic beam splitter) mirror 410, a tube lens 415, and an emission filter 417. While the microscope system is shown comprising a first and second optical arrangement, it is to be understand that one, or a plurality (e.g., three or more) of optical arrangements may be included in the microscope system.

The excitation light may induce an emission light 416 from the target object. A light 416 from the target object 414 can be directed from/by the light conduit 412 to an image capture circuit 418. The microscope system 400 may be configured to direct light from and capture image data for a field of view 426. The microscope system can additionally comprise one or more optical elements (e.g., filters) 413 configured to prevent or reduce chromatic aberrations. In some embodiments, the microscope system 400 can be configured to support wireless communication (e.g., via a wireless adapter). The wireless communication can be via a radio frequency or optical link. For example, one or more images captured by the microscope can be wirelessly communicated to an external processor communicatively coupled to a memory with instructions to receive the one or more images.

The microscope may comprise a housing. The housing may comprise the dimensions 420 and 422. The various elements illustrated in FIG. 4 may be integrated within the housing. The housing may partially or completely enclose the various elements. Optionally, some of the elements may be configured to be coupled to, but external to the housing. For example, light sources, or components of the image capture circuit may be external to the housing. Alternatively, the light sources, or components of the image capture circuit can be partially enclosed by the housing. Optionally, one or more elements can form part of the outer surface of the housing.

Various reagents may be suitable for coating the light conduit. The reagents may comprise drugs, viruses, cells, or other biological materials. The reagents may comprise adeno-associated viruses (AAV). The AAV may encode fluorescent calcium indicators, opsins, fluorescent voltage indicators, static fluorescent reporters, transgenes, or combinations thereof. The AAV may comprise transgenes that are configured to reduce inflammatory response, immune response, or provide therapeutic benefit at the target site. The reagents may comprise nucleotide sequence or encoded genes of a virus. The reagents may comprise one or more types of AAV. The concentration of the AAV deposited on the surface of the light conduit can be between 10¹² to 10¹⁴ viral genome/mL, 10¹¹ to 10¹⁵ viral genome/mL, 10¹⁰ to 10¹⁶ viral genome/mL, or 10⁹ to 10¹⁷ viral genome/mL. The one or more reagents may be dry.

The reagents coating the light conduit may further comprise secondary reagents. The secondary reagents may comprise polymers. The polymers may comprise naturally derived polymers. The polymers may comprise synthetic polymers. The secondary reagents may comprise polymeric conjugates, micelles, hydrogels, microparticles, nanoparticles, microspheres, or nanospheres. The secondary reagents may comprise one or more polymeric materials. The secondary reagent may be suitable for dispersing and coating the reagent on the implantable light conduit.

In some instances, the light conduit may be coated with secondary reagents that may not adversely impact or maintains the properties of the reagents and the function of the light conduit. The reagents may comprise of agents that do not interfere with the optical clarity and/or quality of the light conduit while in use. The secondary reagents may be configured to protect drugs, viruses, cells, or other biological materials from degradation during storage. The degradation may be measured by reduction in efficiency after in vivo implantation of the reagent-coated implantable light conduit.

The coating of secondary reagents on the surface of the light conduit may be degradable. The secondary reagents may be photodegradable, biodegradable, or degradable by electrochemical methods, electrical signal, including electrical current, change in pH, change in temperature, or change in salinity. The secondary reagents may modulate the release of the reagent from the surface of the light conduit. The secondary reagents may be degradable by more than one method. In some instances, signals may be transmitted through the light conduit to the target tissue to modulate the degradation of the secondary reagents and the release of the reagents to the target site. The secondary reagents may be configured to protect drugs, viruses, cells, or other biological materials. The secondary reagents may be configured to desorb the reagents, such as drugs, viruses, cells, or other biological materials, from the surface of the light conduit at a controllable rate. The capability to control the release of the reagents from the surface of the light conduit provides a control over the spatial as a well as temporal delivery of the reagents to the target site. The coating of reagents may be applied in multiple layers to provide a release of each layer at separate time points. The release of each layer may be further triggered by transmitting a signal through the light conduit. The secondary reagents in each layer of coating may be chosen to control the release or desorption rate of the reagents within the layer.

The light conduit may be coated with reagents that may be non-cytotoxic. The reagents coating the light conduit may not negatively affect or maintain the viability and function of tissues and cells adjacent to the light conduit. The polymer in the reagent coating the light conduit may be biocompatible and may avoid irritation to body tissue.

The secondary reagents coating the light conduit may comprise alginate, cellulose, collagens, chitins, chitosan, hyaluronic acid and its derivatives, fibronectin, fibrin, silk fibroin, gelatin, polylactic acid (PLA), polyglycolic acid (PGA), polylactic acid-polyethylene oxide copolymers, polyethylene glycol (PEG) and its derivatives, polyethylene glycol diacrylate (PEGDA), polycaprolactone (PCL), poly(methacrylate), polysaccharides, polystyrene (PS), polyvinyl esters, poly (lactide-co-glycolide) (PLGA), poloxamer F68, poloxamine 908, silicones, polysiloxanes, polyurethanes, natural or synthetic peptides, dendrimers, or combinations thereof. The reagents coating the light conduit may comprise a hybrid polymer. Optionally, the hybrid polymers may comprise sections with crosslinking groups. The crosslinking group may crosslink by application of light, electrical signal, electrical current, change in temperature, change in pH, or change in salinity. The hybrid polymers may comprise sections with cleavable groups. The cleavable groups may be photocleavable groups. The photocleavable group may comprise 4,5-dimethoxy-2-nitrobenzyl (DMNB). The cleavable group may cleave by application of light, electrical signal, electrical current, change in temperature, change in pH, or change in salinity.

The composition of the secondary reagents coating the light conduit may be chosen to modulate the release of the reagents from the surface of the light conduit after implantation. The composition of the secondary reagents may comprise light responsive reagents. The light responsive reagents degrade upon application of light and enhance release of the reagents from the surface of the light conduit. The responsive reagents that degrade upon application of a stimulus, such as light, electrical signal, temperature, or chemicals, may enable controlled release of the AAV, therapeutics, or biological materials in the reagents.

The light conduit may be coated with secondary reagents that enhance coating of the reagents on the light conduit. The secondary reagents may comprise sugars and/or alcohols. The secondary reagents may comprise sorbitol. The secondary reagents may comprise a buffer compatible with the reagents. Sodium chloride, phosphate, citrate, or other tonicity agents may be optionally used to adjust tonicity.

The light conduit may be coated with secondary reagents that enhance the stability of the reagents coating the light conduit during storage. The secondary reagents may comprise glycerol. The secondary reagents may comprise a cryoprotectant agent. The secondary reagents may comprise an agent to protect against thermal damage. The secondary reagents may comprise a humidity controlling agent. The secondary reagent may comprise agents to protect from oxidative damage. The agent to protect from oxidative damage may comprise anti-oxidants or reactive oxidative species scavengers.

The light conduit may be coated with reagents that have a viscosity suitable for the coating method used. The reagents may be flowable under ambient conditions. The reagents may comprise agents that enhance the viscosity of the reagent. The reagents may comprise cellulose-based polymers, polyoxyethylene-polyoxypropylene triblock copolymers, dextran-based polymers, polyvinyl alcohol, dextrin, polyvinylpyrrolidone, polyalkylene glycols, alginate, chitosan, collagen, fibronectin, gelatin, or hyaluronic acid, or any combinations thereof. The viscosity-enhancing agents may comprise gelling agents and suspending agents. The increase in viscosity of the reagents may allow for application of a thicker layer of coating.

The light conduit may be coated with the reagents described herein. Optionally, the reagents may be coated over any portion of the light conduit, e.g., the distal portion, the proximal portion, the body, etc. In some instances, the reagents may be coating over the entirety of the light conduit. Alternatively, the reagents may be coated over a portion of the light conduit (e.g., over only the distal portion, the body, the proximal portion, or the imaging surface). Optionally, the reagents may be coated over a limited potion of the light conduit, for example, over a small portion of the imaging surface. The portion of the light conduit that interfaces with the light delivery system may not be coated with the reagents. The coating of reagents may evenly cover the surface of the light conduit in its entirety. The coating of reagents may evenly cover a portion of the surface of the light conduit. The coating of one or more reagents may be deposited in a pattern on the surface of the light conduit.

The reagents may coat at least 10%, 20%, 30%, 40%, 50%, 60%, 70%, 80%, or 90%, or 100% of the surface area of the light conduit. The reagents may coat at least 10%, 20%, 30%, 40%, 50%, 60%, 70%, 80%, or 90%, or 100% of the surface area of the distal portion of the light conduit. The reagents may coat at least 10%, 20%, 30%, 40%, 50%, 60%, 70%, 80%, or 90%, or 100% of the surface area of the body of the light conduit. The reagents may coat at least 10%, 20%, 30%, 40%, 50%, 60%, 70%, 80%, or 90%, or 100% of the surface area of the proximal portion of the light conduit. The reagents may coat at least 10%, 20%, 30%, 40%, 50%, 60%, 70%, 80%, or 90%, or 100% of the surface area of the imaging surface of the light conduit.

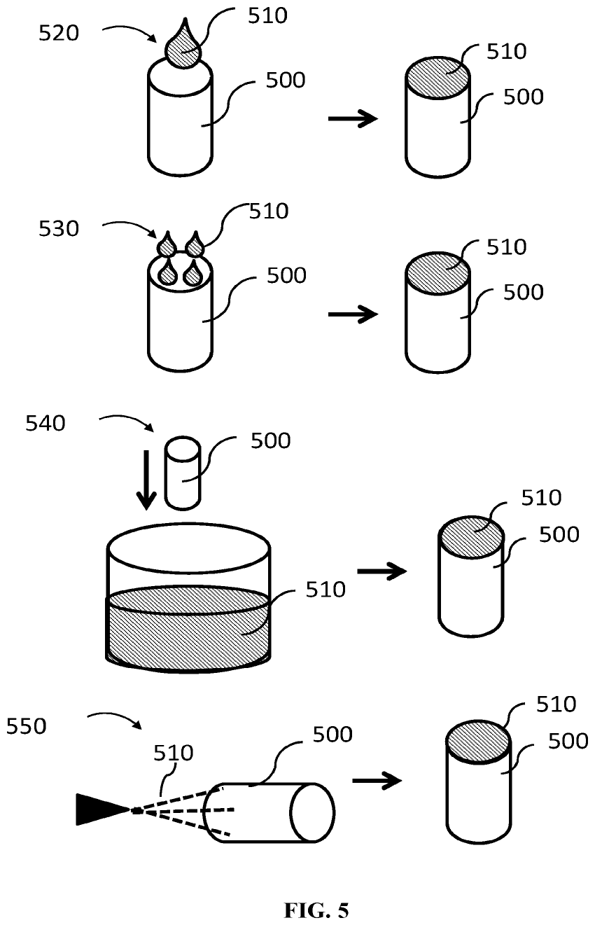

The light conduit may be coated with reagents by manual coating, direct deposition, dip-coating, spraying, impregnating, vapor deposition, polymerization, laminating, pressing, brushing, swabbing, electrostatic deposition, vacuum evaporation, or other chemical methods of immobilization to surfaces. More than one coating method may be used to coat the light conduit. Vapor deposition method may be used with reagents comprising vaporizable, organic compounds. Vapor deposition may be plasma-enhanced chemical vapor deposition polymerization (PE-CVD). The reagents may be coated on the light conduit by applying light, UV, laser, gas, electric field, temperature, or combinations thereof to the reagent. FIG. 5 shows examples of methods of coating the light conduit 500 with the reagents 510, including a direct deposition by a single droplet 520, direction deposition by an array of droplets 530, dipping 540, and spraying 550.

The light conduit may be coated with the reagents by dip coating. The light conduit would be dipped or placed into a volume of the reagents for a predetermined length of time. Afterwards, the reagents on the surface of the light conduit may be dried, cured, or polymerized. Coating by dipping may facilitate coating of a large surface area in a relatively short amount of time and be conducive for coating at a manufacturing scale. The thickness of the deposited layer may be controlled by the rate of withdrawal of the light conduit from the dipping solution. Alternatively or in combination, the thickness of the deposited layer may be controlled by the viscosity of the dipping solution.

The light conduit may be coated with the reagents by direct deposition. The deposition of the reagents to the surface of the light conduit may be performed by commercially-available technologies for precision dispensing and depositing nanoliter-microliter scale volumes. The deposition of the reagents to the surface of the light conduit may be controlled for precise volume control.

The deposition of reagents on the light conduit may be controlled for even application across the entire surface of the imaging surface of the light conduit in a uniform thickness. The reagents may be deposited for variable thickness or a gradient of thickness of coating across the imaging surface. The variable thickness or gradient of thickness may be achieved by gravity. The reagents may be deposited across a portion of the imaging surface. The reagent deposited across a portion of the imaging surface may be spread across a larger portion of the imaging surface subsequent to deposition, by gravity, spinning, tilting, or movement of the light conduit.

The volume of the reagent that is directly deposited to the surface of the light conduit may range from 1 nL to 1000 nL. The volume of the reagent that is directly deposited to the surface of the light conduit may range from 1 μL to 1000 μL. The volume of the reagent that is directly deposited to the surface of the light conduit may be equal to or greater than 0.5 nL, 1 nL, 5 nL, 10 nL, 50 nL, 100 nL, 500 nL, or 1000 nL. The volume of the reagent that is directly deposited to the surface of the light conduit may be equal to or greater than 10 μL, 50 μL, 100 μL, 500 μL, or 1000 μL. The volume the reagent may be repeatedly applied. The total volume of the reagent applied to the light conduit may be about 2000 nL. The total volume of the reagent applied to the light conduit may be equal to or greater than 0.5 nL, 1 nL, 5 nL, 10 nL, 50 nL, 100 nL, 200 nL, 300 nL, 400 nL, 500 nL, 1000 nL, 2000 nL, 3000 nL, 4000 nL, 5000 nL, 6000 nL, 7000 nL, 8000 nL, 9000 nL, or 10000 nL.

Coating the light conduit with the reagents comprises contacting the light conduit with an effective amount of the reagent for a predetermined time. The predetermined time may be at least 1 minute, 5 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, or 12 hours. The predetermined time may be at most 1 minute, 5 minutes, 10 minutes, 15 minutes, 20 minutes, 25 minutes, 30 minutes, 45 minutes, 1 hour, 2 hours, 3 hours, 4 hours, 5 hours, 6 hours, 7 hours, 8 hours, 9 hours, 10 hours, 11 hours, or 12 hours.

The reagents may be applied to the light conduit with the reagent at various conditions, including temperatures, humidity, and pressure. The reagents may be applied to the light conduit at a first temperature throughout the entire coating process. The reagents may be applied to the light conduit at a first temperature and dried at a second temperature. The first and second temperatures may be different. The second temperature may be higher than the first temperature. A higher second temperature may facilitate drying of the reagent on the surface. The temperatures may be modulated during the coating process to enhance the adherence of the coating to the surface. The reagents may be applied to the light conduit at room temperature. The first temperature may be equal to or higher than 0° C., 4° C., 10° C., 15° C., 20° C., 25° C., 30° C., 35° C., 37° C., 40° C., 45° C., or 50° C. The first temperature may be equal to or lower than 0° C., 4° C., 10° C., 15° C., 20° C., 25° C., 30° C., 35° C., 37° C., 40° C., 45° C., or 50° C. The second temperature may be equal to or higher than 0° C., 4° C., 10° C., 15° C., 20° C., 25° C., 30° C., 35° C., 37° C., 40° C., 45° C., 50° C., 60° C., 70° C., 80° C., or 90° C. The second temperature may be equal to or lower than 0° C., 4° C., 10° C., 15° C., 20° C., 25° C., 30° C., 35° C., 37° C., 40° C., 45° C., 50° C., 60° C., 70° C., 80° C., or 90° C.

The reagents may be applied to the light conduit at a first humidity and dried at a second humidity. The first and second humidities may be the same. The first and second humidities may be different. The second humidity may be lower than the first humidity. The first humidity may be equal to or higher than 0%, 10%, 20%, 30%, 40%, 50%, 60%, 70%, 80%, 90%, or 100% relative humidity. The first humidity may be equal to or lower than 0%, 10%, 20%, 30%, 40%, 50%, 60%, 70%, 80%, 90%, or 100% relative humidity. The second humidity may be equal to or higher than 0%, 10%, 20%, 30%, 40%, 50%, 60%, 70%, 80%, 90%, or 100% relative humidity. The second humidity may be equal to or lower than 0%, 10%, 20%, 30%, 40%, 50%, 60%, 70%, 80%, 90%, or 100% relative humidity.

The reagents may be applied to the light conduit at a first pressure and dried at a second pressure. The first and second pressures may be the same. The first and second pressures may be different. The second pressure may be lower than the first pressures pressure. The first pressure may be equal to or higher than 0 atm, 0.5 atm, 1 atm, 1.5 atm, or 2 atm. The first pressure may be equal to or lower than 0.5 atm, 1 atm, 1.5 atm, or 2 atm. The second pressure may be equal to or higher than 0 atm, 0.5 atm, 1 atm, 1.5 atm, or 2 atm. The second pressure may be equal to or lower than 0.5 atm, 1 atm, 1.5 atm, or 2 atm. Coating at pressures different from ambient pressure may be performed in a pressurized vessel.

The reagents may be covered with an outer layer, wherein the outer layer protects the reagents coating the light conduit. The outer layer may be chosen to impart a desired characteristic. The outer layer may comprise a hydrogel. The outer layer may have a low surface energy. The outer layer may have a high surface energy. The surface energy may influences cell adhesion. The outer layer may be biodegradable. The outer layer may be chosen to control the rate of hydrolysis of the outer layer. The outer layer may be chosen to control the release kinetics of the reagent from the light conduit.

The light conduit may be covered with one coating layer. The light conduit may be covered with more than one coating layer. The light conduit may be covered with at least 5, 10, 15, or 20 coating layers. The light conduit may be covered with different coating layers. The coating may comprise a first layer and a second and/or subsequent layer that contain different reagents. The first layer and the second and/or subsequent layer may contain identical reagents having different concentrations. The first layer and the second and/or subsequent layer may contain identical reagents having identical concentrations.

A coating of the reagents on the light conduit may have a mean thickness of 5 nm to 1000 nm. A coating of the reagents on the implantable light conduit may have a mean thickness of equal to or less than 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. A coating of the reagents on the implantable light conduit may have a mean thickness of equal to or greater than 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, or 1000 nm. Thicker coating may release over a longer time frame than a thinner coating. Thickness of the coating of the reagents on the light conduit may be chosen to control the release rate and overall release time of the reagents.

The surface of the light conduit may be subjected to a pre-treatment before coating with the reagents. The pre-treatment comprises roughening, oxidizing, sputtering, plasma-deposition, or priming, or combinations thereof. The coating of the reagents to the light conduit may be enhanced by pretreatment of the surface in the oxygen plasma.

Coupling of the one or more reagents to the light conduit may be enhanced. As one example, the surface may comprise pores. The imaging surface may comprise pores. The pores may provide openings to which the one or more reagents adhere to. The pores may provide a greater surface area to which the one or more reagents may adhere to and allow for more of the one or more reagents to be coated on to the light conduit.

The light conduit may be processed to introduce pores or textures to its surface. The pores may be generated via etching the surface. The surfaces of light conduit may be textured prior to depositing the reagents. The implantable light conduit surface may be textured uniformly with surface irregularities, including pores, dimples, spikes, ridges, grooves (e.g., microgrooves), roughened texture, surface grain, strips, ribs, channels, ruts. The light conduit may appear frosted and become more translucent when deployed to the target site and in contact with the target site tissue. The light conduit may appear translucent and become more transparent when in contact with the target site tissue. The texture may be formed by any suitable methods, for example, by etching, chemical etching, molding, roughening with abrasives, electrical means, thermal means, laser etching, or other known processes. The textured surfaces may enhance the coating of the reagent to the light conduit. The surface irregularities on the surface of the light conduit may enhance the retention of the reagent on the surface of the light conduit. The depth of the surface irregularities can range from about 10 nm to about 2000 μm. The depth of the surface irregularities may range from about 10 nm to about 100 μm. The depth of the surface irregularities may range from about 10 nm to about 10 μm. The depth of the surface irregularities may range from about 10 nm to about 1 μm. The depth of the surface irregularities may range from about 10 nm to about 100 nm.

The reagents may be encapsulated before coating the light conduit. The reagents may be encapsulated in polymer before coating the light conduit. The reagents may be encapsulated in a polymer and further comprise secondary reagents. The encapsulated reagents may be coated on to the surface of the light conduit alone or with a secondary reagent. The encapsulated reagents may be mixed with a secondary reagent and coated on to the surface of the light conduit. The encapsulated reagents may coat the surface of the light conduit and further coated with secondary reagents onto the surface of the light conduit. The encapsulation may maintain the stability or shelf-life of the encapsulated reagents. The encapsulation may reduce the degradation of the encapsulated reagents. The encapsulation may improve the ability to handle the encapsulated reagents in the various steps to prepare and use the reagent coated light conduit. The encapsulation of the reagents may also provide another way to control the release of the reagents from the light conduit. The reagents may be pre-treated before coating the light conduit.

The coating process of the reagents to the light conduit may comprise one or more coating cycles. Each coating cycle may comprise multiple steps. These steps within a cycle may comprise application of the reagents to the surface, and drying or curing or polymerizing of the reagents on the surface. These steps may optionally include preparing the surface of the light conduit prior to application of the reagent. Each step may be performed at a predetermined condition, which may include (contact or drying/curing) time, temperature, humidity, and pressure. The reagents for a cycle may comprise a predetermined concentration of each component and a predetermined application volume. Each cycle may have different reagents or same reagent as a previous or subsequent cycle. Each cycle may have one or more reagents applied to the surface. In some embodiments, multiple reagents in one cycle may be applied to the same area or on different areas of the surfaces. In some embodiments, multiple reagents in a cycle may be applied in a pattern as to coat with one or multiple viruses, or reagents, onto the lens surface. The light conduit with patterned coating may enable spatially targeted delivery of viruses, or reagents, to different sub-regions of the imaging field-of-view and brain tissue.

The coating process may take place under conditions where the reagent is exposed to minimal air particulates or other sources of contamination. Coating process may be performed under aseptic or sterile conditions.

The concentration of each component of the reagent may be determined experimentally. For example, the desired concentration of each type of virus may be determined for each brain region and cell type targeted. The desired concentration of virus may vary depending on the target site. For example, the optimal concentration of virus encoding GCaMP (calcium indicator) may be determined for a specific brain region and cell type. In this example, a serial dilution of virus is delivered across several subjects (mice), and in vivo imaging and post-mortem histology are performed to quantify calcium imaging performance metrics and expression levels.

In some aspects, described herein is a method for directing light to a target site of a subject, bringing a light conduit into contact to the target site of the subject, wherein the light conduit may be coated with one or more reagents that can be released from the light conduit. The release of the reagents from the light conduit delivers the reagents to the target site of the subject, wherein delivery of the reagents may be close in distance to the surface of the light conduit. The method further comprises imaging the target site of the subject or stimulating the target site of the subject by light. The method may involve implanting the light conduit in the target site of the subject. Imaging the target site may comprise directing light to the target site via the light conduit, inducing a response from the target site, and directing the response with aid of the light conduit to a detector.

The light conduit may need to be prepared before implantation. For instance, if the light conduit was stored at −80° C., the light conduit may need to be brought up to room temperature or physiological temperature prior to implantation.

In some aspects, the implantation of virus-coated lenses into the brain can be conducted following existing, standard Inscopix protocols for implantation (Gulati et al., 2017; Resendez et al., 2016). Minor modifications to the protocol may be performed. For instance, depending on the strength of adherence between the virus and lens surface, it may be necessary to minimize lens contact and contact time with biological fluids (e.g. cerebrospinal fluid, blood) prior to final site of implantation.

Virus-coated light conduit can be envisioned for AAV encoding other relevant transgenes including opsins (e.g. Channelrhodopsin), voltage indicators and static fluorescent reporters (e.g. GFP). Virus-coated glass windows may be envisioned that are placed directly onto the surface of the brain (e.g. cortex) and act as a cranial window for in vivo calcium imaging.

Implanting the light conduit and delivering the one or more reagents can be accomplished via a single penetration into the subject. Implanting the light conduit and delivering the one or more reagents is accomplished in a single surgery. The success rate of obtaining co-registration between the one or more reagents in the target site and the light conduit can be equal to or more than 95%, 90%, 80%, 70%, 60%, or 50%.

The release of the reagents from the surface of the light conduit may occur passively. The release of the reagents from the surface may occur by diffusion. The release of the reagents from the surface may occur with aid of light. Light may cleave or degrade the reagent coating to enhance the release of the reagent from the surface. The release of the reagents from the surface may occur with aid of electrochemical methods or stimulus. The electrochemical stimulus may cleave or degrade the reagent coating to enhance the release of the reagent from the surface. The release of the reagents from the surface may occur with aid of electrical stimulus, such as electrical current. The electrical stimulus may cleave or degrade the reagent coating to enhance the release of the reagent from the surface.

The coating of the reagents on the light conduit may be degradable. The reagents may be released from the coating over at least 1, 2, 3, 4, 5, 6, 7, 8, 9, 10, 20, 30, 40, 50, or 60 minutes when the light conduit is deployed in a subject. The release of the reagents may be triggered by degradation of the coating by application of tissue fluids upon implantation. The reagents may be released from the coating over at least 1, 2, 3, 4, 5, 6, 7, 8, 9, 10, 20, 30, 40, 50, or 60 minutes after a signal is transmitted through the light conduit to trigger the release of the reagents. The release of the reagents may be triggered by degradation of the coating by application of light, electrical signal, chemical change, temperature change, pH change, or salinity change after implantation. The release of the reagents may be delayed 1 hour, 2 hours, 3 hours, 4 hours, 5 hours, 6 hours, 7 hours, 8 hours, 9 hours, 10 hours, 11 hours, or 12 hours after the procedure deploying the light conduit. The release of the reagents may be delayed for at least 1 day, 2 days, 3 days, 4 days, 5 days, 6 days, or 7 days after the procedure deploying the light conduit. The reagents coated on the light conduit may be chosen to control its release, where the degradation of the coating of the reagents may be triggered by a signal. The release of the reagents may be triggered at multiple time points, where one layer of the multiple coating layers with different degradation mechanisms may be released at each time point. Each layer may comprise different reagents targeted for assessing different cell activities or different cell types.

The release of the reagents may be substantially restricted to a distance close to the surface area of the light conduit. The reagents may be concentrated to a region close to the light conduit at the target site. This enables the reagents to be delivered to a targeted location and reduce off-target exposure of reagents to surrounding brain tissue. The accuracy of co-registration of the reagent and the light conduit at the target site allows for more accurate imaging, targeting, and/or modulating of the target site. The reagents may travel less than 1 μm from the surface of the light conduit. The reagents may travel less than 10 μm, 50 μm, 100 μm, 500 μm, or 1000 μm from the surface of the light conduit. The distance the reagents travel may be affected by the rate of uptake of the reagents by cells in the target site, release rate of the reagents from the light conduit, and density of the target site tissue.

The reagent may remain adherent to the surface of the light conduit during storage and may be released from the surface upon implantation at the target site. The reagent coating may release from the surface upon implantation of the light conduit and diffuse into the surrounding brain tissue.

The reagent coating described herein are stable in various storage conditions including refrigerated, ambient, and accelerated conditions. The stability is measured for the reagent coating present on the light conduit. In some aspects, stable used herein refers to a reagent coating maintaining its adherence to the surface of the light conduit. It may be important for the reagent coating to properly adhere to and be stable on the surface of the light conduit for the activity of the reagent after implantation.

The light conduit may be stored in a controlled environment. The controlled environment may be a temperature controlled environment. The storage temperature of the reagent-coated light conduit may have an effect on viral infection and transgene expression. AAV may be stored long-term within air and water-tight tubes at −80° C. At this temperature, viral infectivity can diminish very slowly, and AAV can be stable for several years. Storage at warmer temperatures, such as −20° C., 4° C. or at room temperature, may affect the viral infectivity. AAV can be stable at a range of temperatures, and there may be a wide range of potentially acceptable temperatures for storage of virus-coated lenses.

Stable as used herein refers to a reagent having less than 10% loss of reagent activity upon use of the light conduit at the end of a given storage period and at a given storage condition. Reagent activity can be assessed by known testing method. The reagent activity may be infectivity of the virus in the reagent. The reagent activity may be the level of transgene expression. The reagent activity may be activity of a biological material in the reagent. The stable composition has less than 30%, 25%, 20%, 15%, 10%, 9%, 8%, 7%, 6%, 5%, 4%, 3%, 2%, or 1% loss of reagent activity at the end of a given storage period. The stable composition has zero loss of reagent activity at the end of a given storage period and at a given storage condition. The given storage period may be equal to or greater than 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 23 months, or 24 months. The given storage condition may comprise humidity of equal to or less than 10%, 20%, 30%, 40%, 50%, 60%, 70%, 80%, 90%, or 100% relative humidity. The controlled storage environment may comprise humidity between 0% and 50% relative humidity, 0% and 40% relative humidity, 0% and 30% relative humidity, 0% and 20% relative humidity, or 0% and 10% relative humidity. The controlled storage environment may comprise temperatures of −100° C., −80° C., −20° C., 4° C., about 25° C. (room temperature), or 40° C. The controlled storage environment may comprise temperatures between −80° C. and 25° C., or −100° C. and 40° C. The controlled storage environment may protect the light conduit from light or from mechanical damage. The controlled storage environment may be sterile or aseptic or maintain the sterility of the light conduit. The controlled storage environment may be aseptic or sterile.

The coating of the light conduit with reagents may take place under sterile or aseptic conditions, and the sterile, coated light conduit may be sealed in sterile packaging. The reagent and the light conduit may be sterile before coating process and remain sterile after the coating process.

The reagent may be compatible with sterilization. The coated implantable light conduit may be packaged and then sterilized. The sterilization comprises autoclaving, ethylene oxide sterilization, gamma irradiation, X-ray irradiation, or electron beam irradiation. The reagent for coating may be heat-resistant to at least 200° C. to be compatible for sterilization by autoclaving. The reagent may be compatible with ethylene oxide sterilization, gamma irradiation, X-ray irradiation, or electron beam irradiation. The reagent may be compatible with sterilization and does not lead to the degradation of the reagent or the coating of the reagent on the light conduit. The packaging may be selected to be compatible with sterilization and does not lead to the failure of the reagent. In some embodiments, reagent that is compatible with sterilization may have good bonding to the surface of the light conduit.

The reagent-coated light conduits can be packaged to be stored for extended periods of time prior to implantation. The light conduit may be coated with the reagent, and the coated light conduit can be packaged to avoid degradation of the reagent. Packaging may protect the coated light conduit from mechanical damage or thermal damage. The packaging may protect the coated light conduit from contamination of the reagent coating. The coated light conduit may be transported under conditions similar to the storage conditions that result in high stability of the reagent or little loss of reagent activity. The packaging may be configured to provide and maintain sterility of the reagent-coated light conduit. The packaging may be further configured to enable the end-user to pick up the light conduit without compromising its sterility. The reagent-coated light conduits can be compatible with standard manufacturing and shipping operations.

A subject may be a human. A subject may be a mammal. The subject may be a monkey, a mouse, a rat, a pig, a dog, a guinea pig, or a rabbit. The subject may be a non-human primate. The subject may be a marmoset or a macaque. The subject may be a bird. It will be appreciated that the present disclosure would offer benefit to subjects and improve co-registration of reagents with the light conduit.

### EXAMPLES

The invention is further limited by the following non-limiting examples.

### Example 1

### Precision Dispensing and Depositing of Virus on to the Surface of GRIN Lenses

In the following example, microendoscopic GRIN lenses were coated with adeno-associated virus (AAV) encoding a fluorescent calcium indicator (GCaMP6) for performing in vivo calcium imaging in a mouse model. These include seven separate implantations of virus-coated lenses and subsequent in vivo calcium imaging and post-mortem histology in the cortex or striatum of wild-type mice. Optionally, the GRIN lens may be integrated with a microscope baseplate.

Viral infection and transgene expression in the brain can be dependent on several factors, including, but not limited to, the concentration/titer and volume of virus delivered to the brain, the temperature to which the virus is exposed prior to delivery to the brain and the virus' overall purity. The concentrations and volumes that are applied to the surface of the lens may be precise, under clean and temperature-controlled conditions, as to enable consistent results. It may be important that the virus be applied such that it evenly covers the entire intended surface area of the imaging face of the lens.

As a proof-of-concept of the disclosure, virus was applied to lenses, including both straight probes and prism probes, at ambient, room temperature as follows:
Step 1: Lens was held in place with the imaging face of the lens facing upwards.
Step 2: 2 μl aliquot of AAV (˜1E+13 vg/mL) was thawed on wet ice and briefly spun down in microcentrifuge. The entire volume was pulled up into a pipette.
Step 3: A droplet (˜250-750 nL) was pipetted onto the imaging face of the lens and allowed to partially dry (˜5-15 minutes). This deposited viral particles on to the surface of the lens.
Step 4: Step 3 was repeated until the entire 2 μl volume was dispensed and deposited in multiple layers on to the surface of the lens.
Step 5: The virus-coated lens was ready to be implanted into brain or stored for later implantation.

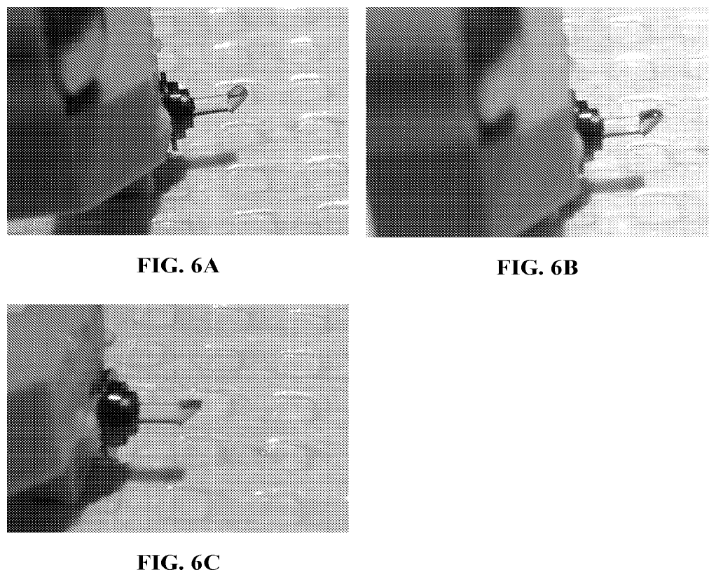

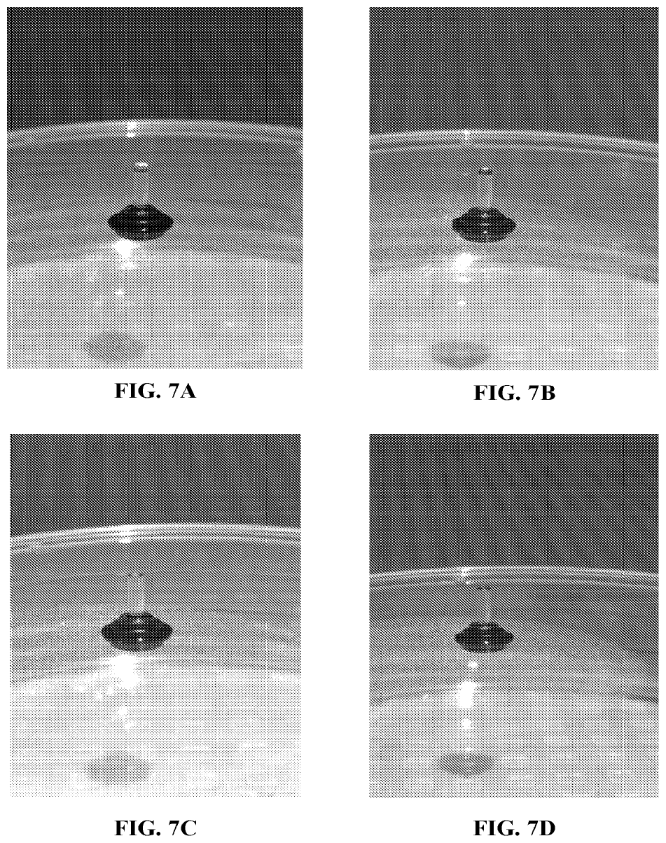

FIGS. 6A-C (for prism probe) and FIGS. 7A-D (for straight probe) show examples of the virus droplets after being applied to the lens surface and over the course of several minutes as the droplet evaporates and viral particles are deposited onto the surface of the lens. FIGS. 6A-C show a prism probe following application of a droplet (˜250-500 nL) comprising virus (AAV1.CaMK2a.GCaMP6f) that was pipetted onto imaging face of the lens, at immediately following application (FIG. 6A), approximately 5 minutes following application (FIG. 6B), and approximately 10 minutes following application (FIG. 6C). FIGS. 7A-C show a straight probe following application of a droplet (˜500-750 nL) comprising virus (AAV9.CAG.GCaMP6m) that was pipetted onto imaging face of the lens, at immediately following application (FIG. 7A), approximately 5 minutes following application (FIG. 7B), approximately 10 minutes following application (FIG. 7C), and approximately 15 minutes following application (FIG. 7D).

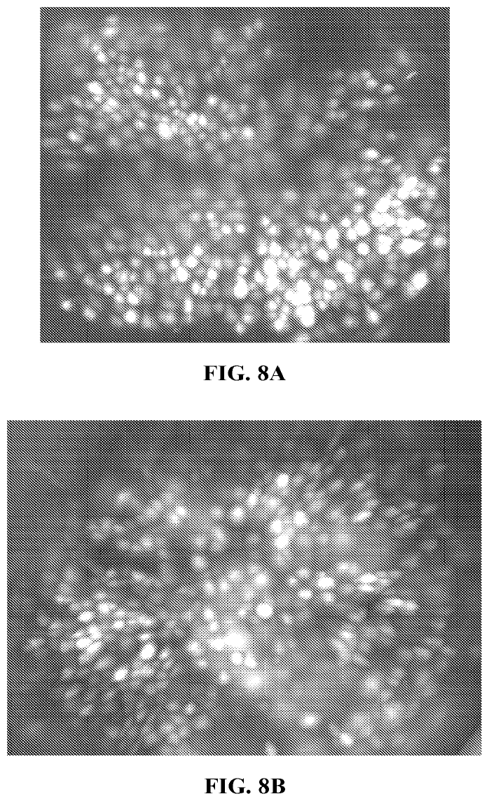

FIGS. 8A-B provides static image representations from a video of cellular calcium dynamics imaged in-vivo ˜3 weeks following prism (FIG. 8A) and straight (FIG. 8B) lens implantation in mice. FIG. 8A shows the maximum intensity merged image of individual cell mixing images resulting from PCA/ICA (Mukamel et al., 2009) of primary somatosensory cortex cellular calcium activity (dynamic fluorescence) obtained in vivo at 20 days after implantation of the virus-coated prism probe lens. FIG. 8B shows the maximum intensity merged image of individual cell mixing images resulting from PCA/ICA (Mukamel et al., 2009) of dorsal striatum cellular calcium activity (dynamic fluorescence) obtained in vivo at 21 days after implantation of the virus-coated straight probe lens.

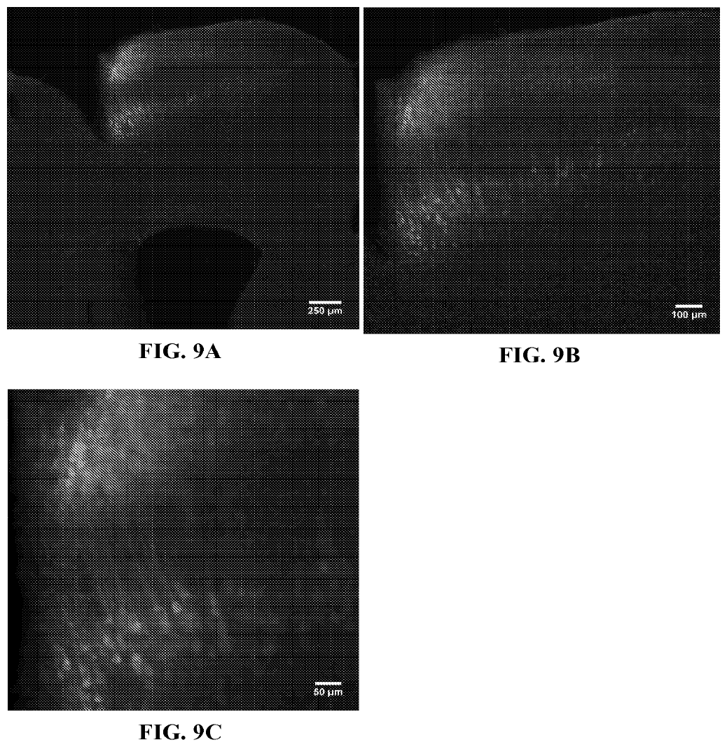

FIGS. 9A-C provides post-mortem histology images of the murine primary somatosensory cortex tissue section at 26 days after implantation of a virus-coated prism probe, from the same case as depicted in FIG. 8A. Primary somatosensory cortex tissue section was stained with DAPI to visualize nuclei, which appeared as blue fluorescent signals, provide a reference for the images. Green fluorescent signals corresponded to native GCaMP6 expression in virus-infected neurons. The tissue section was visualized at low magnification (FIG. 9A), middle magnification (FIG. 9B), and high magnification (FIG. 9C). The presence of green fluorescent signals in the tissue sections indicated extent of viral infection and GCaMP6 expression surrounding the implanted lens.

Within days of lens implantation, viral infection and transgene (e.g. GCaMP6) expression was apparent by imaging through the lens with existing Inscopix miniature microscope technology (single photon fluorescence microscopy). The single surgery/procedure of lens implantation was able to accomplish with one brain penetration both the delivery of the virus and placement of the lens with optimal registration between the two. Virus infection and transgene expression was restricted to the genetically targeted cells positioned immediately in front of the imaging face of the lens, minimizing out of focus fluorescence from cells further away that can reduce overall signal-to-noise for imaging.

The overall success rate for these experiments can be improved over existing standards, and the level of training required to obtain these high success rates is significantly reduced.

### Example 2

### Virus Coated Prism Probe: 4° C. Storage

Toward proof-of-concept and to begin to assess bounds regarding storage conditions, an experiment was performed. 2 μl of virus was applied to the imaging face of a prism probe according to the protocol described in the Example 1. The last droplet of virus was not allowed time at room temperature to dry on to the surface of the lens. The probe was placed inside a plastic petri dish with lid and immediately transferred to the refrigerator for 24-hour storage at 4° C.

After 24 hours of storage, virus-coated lens was removed from the refrigerator and allowed to equilibrate to room temperature prior to implantation into the brain according to standard Inscopix implantation protocol (Gulati et al., 2017) as described in Example 4. Successful viral infection and transgene (GCaMP6) expression was confirmed in the days and weeks following implantation via in-vivo calcium imaging with the Inscopix nVista microscope.

### Example 3

### Virus Coated Prism Probe: Room Temperature Storage

Toward proof-of-concept and to begin to assess bounds regarding storage conditions, an experiment was performed. 2 μl of virus was applied to the imaging face of a prism probe according to the protocol described in Example 1. The entire 2 μl volume of virus had time to dry on to the surface of the lens. The probe was placed inside a plastic petri dish with lid and remained for 24-hour storage at ambient, room temperature.

After 24 hours of storage, virus-coated lens was implanted into the brain according to standard Inscopix implantation protocol (Gulati et al., 2017). Successful viral infection and transgene (GCaMP6) expression was confirmed in the days and weeks following implantation via in vivo calcium imaging with the Inscopix nVista microscope.

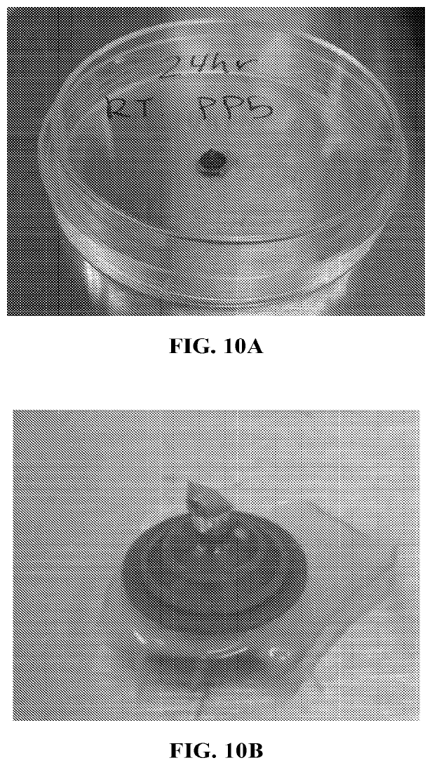

FIGS. 10A-B show the images of 24 hour storage of virus-coated prism probe lenses stored inside a plastic petri dish at room temperature at low magnification (FIG. 10A) and higher magnification (FIG. 10B).

### Example 4

### Protocol for Cortical Imaging Using Cranial Window

The implantation of virus-coated lenses into the brain can be conducted following existing, standard Inscopix protocols for implantation (Gulati et al., 2017; Resendez et al., 2016).

In this example of a protocol for cortical imaging using a cranial window, anesthetize a mouse using isoflurane (5% for induction, 1-2% maintenance in oxygen, flow rate ˜0.5 L/min). After shaving and disinfecting the top of the animal's head, expose the surface of the cranium at the site for cannulation. Open an oval-shaped area approximately 1.2 cm (lateral extent, equally across midline) by 1.5 cm (rostrocaudal extent). Then, remove the periosteum, rinse with ACSF, dry the area, and polish the exposed skull.

Begin to draw a round craniotomy centered over the stereotaxic coordinates of interest with the microdrill, slightly larger than the size of the 3 mm round glass coverslip (cranial window). Create the craniotomy, taking care to not puncture through the skull during drilling. Drill in circles, drawing out the bone island gradually, keeping the drill bit more horizontal than vertical. Every few minutes, be sure to saturate the drilling site with ACSF/sterile saline and then wick it away with a cotton swab. This serves both to wash away bone dust, as well as cool the brain tissue.

Remove the bone island with forceps when a part of it is loosening from the surrounding skull. As soon as the bone is removed, keep the exposed dura and brain tissue moist with Gelfoam or a drop of ACSF/sterile saline.

Apply a clean, dry glass coverslip that has been coated with reagents (virus) over moist, exposed brain tissue, and set the coverslip directly over the brain. Installing the coverslip too high above the brain surface will drastically reduce the ability to see neural signals due to the focus range of the nVista system. A drop of ACSF/saline on the brain tissue may be helpful before placing the coverslip down or shifting the coverslip. Apply gentle pressure to create a tight seal and fill any air bubbles in with the addition of a little ACSF/saline with a syringe.

While maintaining gentle pressure on the coverslip, use a very thin layer of silicone adhesive (e.g. WPI Kwiksil) to protect exposed brain tissue that is not covered by the glass coverslip if needed. This may not be needed for flat regions of thin skull (e.g. visual cortex), but can be helpful to create a good seal in thicker, curved regions (e.g. prefrontal cortex, motor cortex). If there are air bubbles under the coverslip, add a little ACSF/saline to fill them in.

Continuing the maintenance of gentle pressure on the coverslip, use an adhesive (e.g. gel Loctite 454) to permanently fix the edges of the coverslip to the surrounding skull, taking care not to let any adhesive touch the brain, or to obscure the window with adhesive. Ideally, the coverslip is flush with the surrounding skull.

Wait for the adhesive to set, so that the coverslip is firmly attached to the surrounding skull. Use dental cement or your adhesive of choice to create a thin cap over the rest of the exposed skull. Do not build up the skull cap any taller than the height of the coverslip.

Return the animal to a clean home cage and maintain heating until recovered from anesthesia. Administer analgesics as dictated by animal care guidelines. Singly house your animals to prevent others from damaging the implant site.

After 1-2 weeks of tissue recovery, anesthetize and place the animal in a stereotax, or in an awake head-fixed running wheel setup to check the implant for neural activity using the nVista system.

### Example 5

### Protocol for Implantation of a Reagent-Coated Probe

The implantation of a reagent-coated lens probe or a reagent-coated prism probe into the brain can be conducted following standard protocols for implantation of a lens probe or a prism probe. In this example, the reagent-coated lens probe or the reagent-coated prism probecan be implanted and secured to the skull without a need for a separate injection of the reagent. The reagent coated prism probe may be integrated with a baseplate. Optionally, a GRIN lens may be integrated with the baseplate.

In a mouse model of implantation, the mouse can be anesthetized using isoflurane (5% for induction, 1-2% maintenance in oxygen, flow rate ˜0.5 L/min) and checked for absence of toe-pinch reflex to assess depth of anesthesia. An anti-inflammatory drug (e.g. carprofen or dexamethasone) can be administered, and the mouse can be positioned and secured in the stereotaxic frame. While maintaining the animal's body temperature at 37° C. with a heating blanket, generously dab ophthalmic ointment on the eyes and place a piece of dark paper over the eyes to shield them from the surgery light source used in the procedure. Trim, shave, or remove the fur from the top of the animal's head, and disinfect the area with three alternating washes of 70% ethanol and betadine.

Once shaved and disinfected, the surface of the cranium at the site for lens probe insertion can be exposed. An oval-shaped area approximately 1.2 cm (lateral extent, equally across midline) by 1.5 cm (rostrocaudal extent) can be opened using skin scissors. The periosteum and nearby muscle tissue can be removed using cotton swabs and a scalpel, and rinsed with ACSF. After rinsing, new cotton swabs can be used to dry and polish the exposed skull. Before proceeding to the next steps, check that the periosteum and all nearby muscle are completely removed across the entire exposed skull face by gently probing the edges with a scalpel blade and level the skull.

Then, open a round craniotomy over the stereotaxic coordinates of interest using the micro-drill with burr. The craniotomy diameter can be ensured to be just slightly larger than the lens diameter. Drill gently through the cross section of the skull, holding the drill in a near horizontal position, pausing halfway through to flush the skull with ACSF and suction with a cotton swab, removing all the bone chips and dust from the drilling process. Stop just before the skull is completely thinned. It should be possible to clearly see blood vessels in the brain tissue under the drilled bone. Gently remove the small skull plug with a pair of fine 45-degree forceps. Remove the dura with forceps. Once the brain tissue is exposed, keep the brain tissue moist under a bubble of ACSF and do not let the tissue dry out. A cotton swab saturated in ACSF can also be gently placed on top of the craniotomy so the brain tissue is under pressure and remains moist.

For a very deep region implantation of the lens probe, it may be necessary to create a tract or a pathway for the lens probe within the tissue. To create a tract, a part of the cortex can be aspirated, and a sterile needle or rod of a slightly smaller diameter than that of the lens probe can be inserted slowly into the tissue until reaching ˜60-70% of the targeted implantation depth for the lens probe.

In aspirating the cortical tissue, a 27 G bent blunt needle can be attached to a vacuum line or pump and held in the dominant hand of the person performing the procedure. A syringe with cold sterile saline solution can be used to constantly irrigate the aspiration site while slowly aspirating the tissue in a radially inward motion. For example, a 100 mbar pressure provided by the pump and a ⅛ inch internal diameter tubing connected to a needle holder can be used for aspiration. Aspirate until a shallow part of the cortex is removed, based on the total depth that the lens probe will be inserted into. If aspirating all the way down, leave a thin layer of tissue intact above the region of interest, which may be ˜500-800 μm in a mouse model.

While bleeding can be expected during the aspiration procedure, the bleeding can be managed by controlling it with a small piece of gelfoam saturated with ACSF or sterile saline. Dry cotton swabs can be used to apply gentle pressure and to wick away any initial flooding of moisture and blood. After bleeding is controlled, keep the gelfoam piece moist and leave it in place for several minutes, until the blood clots and active bleeding stops. Some residual blood may be cleared by the brain during the healing process, but solid blood clots should be removed, as these may not be cleared and can obscure the field of view in the tissue.

For implantation of either a fiber or a small diameter lens probe (e.g. 0.6 or 0.5 mm in diameter) without aspiration, keep moist gelfoam over the craniotomy, and proceed to the next step.

If using the ProView™ Implant Kit, attach the stereotax holder arm and ProView™ base holder to the stereotax apparatus, and if desired, attach the nVista microscope along with the ProView™ cuffed lens probe. Follow the ProView™ Implant Kit instructions on using the kit components.

To prepare for data acquisition, connect the nVista DAQ box to the computer and create a project in the acquisition software. Align the ProView™ lens probe such that it is perpendicular to the surface of the brain. If using a prism probe, ensure that the prism angle is adjusted to accommodate the brain's curvature. This step might require some fine correction of the stereotax arm position. Adjust the alignment by staying close to the skull for faster results. If using a lens probe, prepare the bulldog clip and probe, and attach to the stereotax manipulator arm.

Then, gently remove the gelfoam from the craniotomy. Any blood clots should come out relatively cleanly with the gelfoam. If the blood clot does not move, gently remove it with fine forceps when appropriate. The aspiration site should be free of blood clots and active bleeding. Bring the reagent-coated lens probe or the reagent-coated prism probe close to the craniotomy and zero out z when the lens is just below the skull depth. Gradually lower the lens probe or the prism probe into the brain using the manipulator (10 μm deep at a time) until reaching the desired z.

If using the ProView™ Implant kit, bring the lens over the craniotomy and zero out z when the lens is just beyond the skull depth. Turn on nVista and set LED power to about 50% to start, and gradually lower the stereotax arm (10 μm deep at a time). Perform this step over the course of several minutes.

Wait for several minutes to allow the brain tissue to settle around the lens probe or the prism probe. When using a prism probe, any pressure that is created is behind the visualization plane and will not affect the field of view.

Uncap the Kwik-sil tube and mix the silicone elastomers from the double barrel syringe. Using a 25 G needle, apply a very thin layer around the bottom of the protruding lens probe or the prism probe, just enough to cover any exposed brain tissue. The Kwik-sil functions to cover and protect any exposed brain tissue around the base of the lens probe or the prism probe and craniotomy. A rigid-curing adhesive can be used to rigidly fix the lens probe or the prism probe to the skull. When Kwik-sil has cured, usually in ˜3-5 minutes, ensure the skull is dry and, using a 25 G needle, apply Loctite 454 gel adhesive or standard dental acrylate to fix the lens probe or the prism probe to the adjoining skull so that the probe does not move. Be sure to make ample contact with the sides of the protruding lens probe or the prism probe itself, rather than just Kwik-sil around the lens probe or the prism probe. This will ensure a solid, secure implant.

After the Loctite gel glue is fully cured and rigid, carefully remove the bulldog clamp if using a lens probe setup. If using the ProView™ Implant tool, loosen the base holder set screw and remove the nVista microscope, if used. Incrementally move the holder arm of the stereotax in the z direction away from the skull, and the cuffed lens will come free from the base holder. Then, the ProView™ Implant base holder can be removed from the stereotax manipulator arm. A wall of dental acrylic or Loctite may be built up around the lens/prism probe to create a wall in anticipation of the baseplate install step, and to secure the probe to the skull. Wait for the dental acrylic/Loctite glue to cure. A relatively thick mixture of dental acrylic may be created to combat volume shrinking over the course of adhesive curing.

If using a lens probe or the prism probe, cover the end protruding out of the skull using the inverted tip of a cut PCR tube to prevent physical damage to, or debris reaching, the imaging face. Affix the PCR tube tip using Kwik-cast and a small amount of Loctite glue. If using a ProView™ lens probe, cover the top of the lens with Kwik-cast. The cuff will protect the edges of the lens probe imaging face. If using an awake head-fixed setup for baseplate install, implant a headbar as part of the cranial cap with the optional skull screws.

Then, return the animal to a clean home cage and maintain heating until recovered from anesthesia. Administer analgesics as necessary by the institutional animal care guidelines. Singly house your animals post-surgery to prevent other animals from damaging the implant site.

After 1-2 weeks of recovery, and time for virus infection and transgene expression, anesthetize and place the animal in the stereotax, or in an awake head-fixed running wheel setup to check the implant for neural activity using the nVista system. If satisfied with the extent of neural activity, the baseplate can be installed when the animal is ready for the nVista system installation. An example of the baseplate installation and imaging is described in Example 6.

### Example 6

### Protocol for Implantation of and Imaging with a Prism Probe

In this example, a procedure for performing chronic fluorescence microscopy with cellular-resolution across multiple cortical layers in freely behaving mice is described. An integrated miniaturized fluorescence microscope paired with an implanted, reagent-coated prism probe can be used to simultaneously visualize and record the calcium dynamics of hundreds of neurons across multiple layers of the somatosensory cortex as the mouse engaged in a novel object exploration task, over several days. This technique can be adapted to other brain regions in different animal species for other behavioral paradigms. Further details of the procedures is described in Gulati, S., Cao, V. Y., Otte, S. Multi-layer Cortical Ca2+ Imaging in Freely Moving Mice with Prism Probes and Miniaturized Fluorescence Microscopy. J. Vis. Exp. (124), e55579, doi:10.3791/55579 (2017).

### Prism Probe Implant Surgery

Using a mouse model, the mouse can be anesthetized and prepared as described in Examples 4 and 5. The anesthetized animal can be mounted in a stereotaxic frame fitted with ear and teeth bars.

Then, prepare for the prism probe implantation. Insert a sterile, reagent-coated prism probe in the lens holder tool and tighten the hex screw with the screwdriver. Seat the microscope in the base holder (the magnets will hold it in place). As described in Examples 4 and 5, prepare the mouse for probe implantation by shaving, removing a portion of the skull, and exposing the brain tissue for implantation. Trim and shave animal's head between the eyes and ears and disinfect the skin with alternate swabs of 70% ethanol and betadine. Expose the skull by incising the skin with a pair of sterile scissors and remove the skin flap and underlying periosteum. Dry and polish the skull with cotton swabs. Ensure adequate removal of surrounding muscle tissue to create a clean, dry, wide bone foundation in preparation for the following steps. Implant skull screws in the contralateral hemisphere to make the implant stable and secure. These may also be useful if choosing to implant a headbar for awake head fixing to ready the animal for experimental imaging sessions. Level the skull and with a marker, mark the AP and ML coordinates for lens insertion. Using a 0.5 mm burr on a microdrill open a round craniotomy, ensuring the craniotomy diameter is just larger than the prism diameter i.e. 1.0 mm in this case. Drill gently while pausing intermittently to flush the skull with sterile PBS and suction it away with cotton swabs. Remove the bone dust that is generated.

Stop drilling right before the skull is completely thinned. Blood vessels should be visible through the thinned bone. Remove the bone plug gently with fine 45° forceps. Remove the dura with #5 forceps. Once the brain tissue is exposed, always keep the tissue moist. Place a cotton swab dipped in sterile saline over the craniotomy. This will also maintain pressure on the tissue.

To alleviate pressure in the brain tissue during insertion of the prism probe, create an insertion tract ahead of time. Attach a straight-edged dissection knife to the electrode holder arm of the stereotaxic apparatus and mount it on the stereotaxic apparatus at an angle such that the knife blade is perpendicular to the curvature of the skull (10° angle in this case). Carefully position the knife above the craniotomy along its anterior medial edge in this case and with the cutting edge facing posteriorly. Zero out the Z-axis when the knife tip touches the pia of the meninges and lower it gradually (in 10 μm/s increments) to a depth at which the prism probe will be inserted. Then move the knife 1 mm posteriorly to create a path for the prism's leading edge. Pause and control for any bleeding that may happen while making the incision with a pre-sterile saline soaked piece of gelfoam. Once the knife is in this position, flush the site with sterile saline and wait until any bleeding subsides. Then slowly retract the knife using the stereotaxic arm micromanipulator in 10 μm/s increments, and place a piece of gelfoam sponge soaked in sterile saline over the incision.

Attach the lens holder (with the sterile, reagent-coated prism probe and microscope) to the stereotaxic manipulator arm at the same angle as the knife in the previous step. Align the prism such that the flat side of the prism is over the incision. Adjust the alignment by staying close to the skull for faster results. Once the prism is at the correct angle, gradually lower it in the brain in 10 μm increments to a final z of 1.1 mm for this probe, starting from the brain surface. The brain tissue will expand around the prism and any pressure that is created is behind the visualization plane and will not affect the field of view.

Cover any exposed tissue around the prism in the craniotomy in a very thin protective layer of elastomer adhesive using a 25 G needle. After the elastomer adhesive is cured (usually in ˜3-5 min) use a 25 G needle to apply some cyanoacrylate adhesive to attach the glass of the prism lens to the adjoining skull over the layer of elastomer adhesive, to prevent the lens from moving inside the craniotomy. Include the edges of the prism probe cuff for better adhesion. Do not get any adhesive on the top face of the implanted prism probe. Once the cyanoacrylate adhesive is cured unscrew the lens holder and carefully remove the microscope. Then slowly retract the stereotaxic manipulator arm to leave the prism probe securely implanted.

Apply a layer of dental acrylic or cyanoacrylate adhesive around the implant to cover all exposed skull surface, up to but not touching the surrounding retracted muscle tissue. Covering a large area of skull with this cranial cap will later help in baseplate attachment. The skin around the implant site should heal on its own around the cranial cap. Do not let adhesive touch any of the surrounding skin or muscle tissue, and do not engulf any skin in the cranial cap. Doing so will irritate the skin, and may result in excessive scratching and potential damage to the implant.

Optionally, if wishing to use an awake head-fixed setup to attach and detach the microscope to an animal's baseplate in experimental imaging sessions rather than briefly anesthetizing or scruffing the animal, implant a headbar in the cranial cap that is compatible with an awake head-fixed setup of choice (not demonstrated in this protocol).

Mix the catalyst and base from a silicone adhesive syringe and put a drop of the elastomer inside the prism probe cuff to cover the probe lens top to prevent any damage and dust from settling.

Remove the animal from the stereotaxic frame and allow for recovery from anesthesia in a warm chamber. Administer ketoprofen (2.5 mg/kg) or carprofen (2.5 mg/kg) subcutaneously, and return the animal to a clean home cage once it is ambulatory. Singly house all subjects to protect the implant and repeat the dose 24 h later.

### Baseplate Attachment for Miniature Microscope Installation

At a predetermined time after implantation of the prism probe, which can be one week to several weeks after implantation, the baseplate for the miniature microscope can be attached to the animal with the implanted probe. A light conduit may be integrated with the microscope baseplate. The light conduit may be, for example, a GRIN lens. First, check for virus expression in the tissue through the implanted prism probe, and attach a baseplate on the skull if the preparation shows cell activity. The microscope will dock on the baseplate during live imaging. Again, prepare the mouse by anesthetizing, protecting their eyes, and providing necessary drugs subcutaneously. Remove the silicone adhesive cap over the surface of the implanted prism probe lens top. Examine the lens probe surface, and clean off any debris gently with lens paper and 70% ethanol to ensure the imaging surface is clean.

Plug the microscope to its DAQ box and connect it to the computer. Open the acquisition software on the computer and connect the microscope. Use the acquisition software for checking neural activity, and for measuring and documenting the field of view settings for future recordings in this subject.

Attach a baseplate to the microscope and fasten the baseplate set screw to hold the baseplate in position, and secure the microscope into the microscope gripper on the stereotaxic micromanipulator arm by the body of the microscope. Attach the gripper to a Newport rod, which can be mounted on the stereotaxic micromanipulator arm. Position the microscope above the prism probe lens using the stereotaxic micromanipulator arm. Visually inspect the orientation by viewing the prism lens from the side and back of the animal stage. The optical axes of both the microscope objective and prism probe lens may be aligned.

Turn on the microscope LED through the software. Evaluate the quality of the microscope alignment by focusing on the top face of the implanted prism probe lens in the acquisition software. When properly aligned, the edges of the prism probe lens top face may be sharp. Adjust the microscope's physical distance above the implanted prism probe using the stereotaxic manipulator arm to obtain the desired focal plane inside the tissue. The optically optimized distance between the microscope objective and implanted GRIN lens can be ˜500 μm. Save a reference fluorescence image once the desired imaging plane is captured. From this point on, do not adjust the position of the microscope, as this will change the location of the imaging plane in the tissue.

Apply adhesive in the next step to permanently fix the position of the baseplate in relation to the cranial cap. The adhesive may experience some volume shrinkage in the following day or two, which can change the focal plane in the tissue. Preemptively account for this by measuring the amount of shrinkage for your adhesive mix and distance ex vivo, then backing the final Z position of the microscope+baseplate by that amount before progressing to the adhesive application step. Use dental acrylic or cyanoacrylate to permanently attach the baseplate to the acrylic cap covering animal's skull, bridging the gap with the acrylic or adhesive. Applying the dental acrylic/cyanoacrylate gradually and curing in multiple stages may minimize the effect of the previously mentioned shrinkage on the final position of the microscope's image plane. Take care while applying dental acrylic/cyanoacrylate to prevent any material from contacting the microscope's objective lens, the set screw, or the microscope body, which will prevent proper operation of the instrumentation later on. While applying the adhesive, do not push on the microscope. Pressure on the microscope or baseplate can cause movement of the microscope objective relative to the prism probe lens, which could result in misalignment or a change of the focal plane in the tissue that would require prompt re-adjustment. Verify that the dental acrylic/cyanoacrylate has cured and hardened by tapping the acrylic with a pair of forceps or syringe tip. Acquire a final reference fluorescence image with the acquisition software.

Release the microscope from the gripper and retract the gripper from the microscope. If cyanoacrylate or another transparent adhesive was used, cover it with black nail polish or a layer of black dental cement to prevent ambient light leakage into the head cap, which can contaminate future images acquired during experiments. At this point, remove the microscope if needed. For separating the microscope from the baseplate, release the baseplate set screw by turning the set screw approximately ½ turn counter-clockwise. Pinch the microscope body while supporting the baseplate and acrylic cap with the other hand, and pull the microscope straight up. Replace it in its storage container. Protect the implanted prism probe with a baseplate cover. This will prevent any dust particles from settling on the lens surface, which can be tricky to clean after the baseplate has been installed. Attach the baseplate cover on the baseplate and advance the set screw by an approximate ½ turn clockwise or until the set screw is flush with the baseplate cover. Do not overtighten.

Remove the animal from anesthesia and monitor in a warm recovery chamber until ambulatory. Return the animal to its home cage. Singly house all animals with implanted baseplates to protect the implant.

### Imaging in a Freely Moving Mouse

Once the animal is ready for imaging, the animal can be interfaced with the imaging system. Prepare the behavioral apparatus, (e.g. Phenotyper, Noldus) by cleaning and disinfecting it and wiping down with 10% bleach solution. Plug the microscope to its DAQ box, and connect it to the computer and launch the acquisition software. Check for availability of file storage space. This space can be on the acquisition computer. Prepare room for the calcium imaging movies. Saving directly from the software to a local hard disk, rather than writing to an external hard drive, can accommodate a high data transfer rate between the microscope and the computer and prevent data loss during recordings.

Anesthetize the animal with isoflurane (5% in oxygen) in an induction chamber to attach the microscope. Alternatively, gently scruff the animal or use an awake head-fixed setup with a headbar if anesthesia is known to interfere with the behavioral paradigm of choice. Remove the baseplate cover by turning the baseplate setscrew counter clockwise and lifting out the baseplate cover. Seat the microscope in the baseplate on the animal. The microscope should snap into place with the aid of the magnets on the baseplate. Advance the baseplate set screw until slight resistance is felt. Do not over tighten the baseplate set screw to prevent damage to the microscope housing. Check the imaging plane in the tissue by acquiring a fluorescence snapshot in the software, and if necessary adjust the focal plane in the tissue by loosening the microscope turret set screw, rotating the microscope turret to adjust the fine focus, then re-tightening the turret housing set screw. Do not force the turret to turn without first loosening the set screw, and do not over tighten the turret set screw.

If conducting a longitudinal study, return to the physical turret position to capture the same field of view. In the hardware, note the number of turret turns, or the physical position of the turret, for each animal imaged with the same microscope for a quick return to the same field of view.

Release the animal carrying the microscope into its home cage or behavioral chamber for acclimation, and await the wearing off of anesthesia if applicable. Prior to the imaging session, the animals can be trained to carry the weight of the microscope using a dummy microscope for several sessions until ensured that wearing the microscope does not interfere with their normal behavior, before starting experimental sessions. Regular handling and training for awake restraint may prevent undue stress to the animals.

Select the acquisition settings to be used to gather data. This includes the frame rate for capturing data (e.g. 20 frame per second (fps), gain of 1, and LED power of 50%). Check the image histogram when selecting the settings to ensure good signal-to-noise ratio (SNR). The numerical aperture for fluorescence collection is 0.35 for the 1 mm prism probe compared to 0.5 for the 1 mm straight probe. Launch the behavioral software and program it to trigger the microscope at the desired imaging recording cycle (e.g. a 4× 5 min ON 2 min OFF). Connect the TTL port on the Noldus IO box to the TRIG port on the DAQ box via a RJ45 to BNC cable.

Place the animal in the behavioral arena if necessary, and start the experiment. After acquiring desired data, re-anesthetize the animal with isoflurane (5% in oxygen) in an induction chamber, or gently awake-restrain the animal. Loosen the baseplate set screw and detach the microscope from the baseplate by gently pulling up the microscope. Replace the baseplate cover and gently tighten the baseplate set screw. Return the animal to the home cage until the next recording session. Use the reference fluorescence images as a guide for subsequent imaging sessions to return to the same field of view.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-149. (canceled)

150. An implantable light conduit comprising:
a distal end comprising a surface configured to be implanted in a subject,
a proximal end; and
an elongate body extending between the distal end and the proximal end,
wherein the surface of the implantable light conduit comprises one or more reagents configured to be delivered to the subject.

151. The implantable light conduit of claim 150, wherein the light conduit comprises one or more members selected from the group consisting of a lens, an optical fiber, and a glass window.

152. The implantable light conduit of claim 151, wherein the light conduit comprises a lens, wherein said lens comprises a gradient-index (GRIN) lens.

153. The implantable light conduit of claim 150, wherein the light conduit comprises a volume equal to or less than 10 cm³.

154. The implantable light conduit of claim 150, wherein the light conduit comprises an electrode.

155. The implantable light conduit of claim 150, wherein the surface comprises pores to which the one or more reagents adhere to.

156. The implantable light conduit of claim 150, wherein the one or more reagents comprise drugs, viruses, cells, or other biological materials.

157. The implantable light conduit of claim 156, wherein the one or more reagents comprise adeno-associated viruses (AAV).

158. The implantable light conduit of claim 157, wherein the AAV encodes one or more members selected from the group consisting of fluorescent calcium indicators, opsins, fluorescent voltage indicators, static fluorescent reporters, and transgenes.

159. The implantable light conduit of claim 158, wherein the AAV encodes transgenes, wherein the transgenes are configured to reduce inflammatory response, immune response, or provide therapeutic benefit at a site of implant.

160. The implantable light conduit of claim 157 wherein a concentration of the AAV deposited on the surface of the light conduit is between 10¹⁰ to 10¹⁶ viral genome/mL.

161. The implantable light conduit of claim 150, wherein the one or more reagents comprise one or more secondary reagents selected from the group consisting of synthetic polymers or oligomers, naturally derived polymers or oligomers, and polymeric conjugates, micelles, hydrogels, microparticles, nanoparticles, microspheres, or nanospheres.

162. The implantable light conduit of claim 161, wherein the secondary reagents are photodegradable or biodegradable.

163. The implantable light conduit of claim 161, wherein the secondary reagents are configured to slow desorption of drugs, viruses, cells, or other biological materials from the light conduit at a controllable rate.

164. The implantable light conduit of claim 163, wherein the one or more reagents evenly cover the surface in its entirety.

165. The implantable light conduit of claim 150, wherein the one or more reagents are dry.

166. The implantable light conduit of claim 150, wherein the one or more reagents are pattern deposited on the surface.

167. The implantable light conduit of claim 150, wherein the body comprises a height of at least 1 mm.

168. The implantable light conduit of claim 150, further comprising a baseplate attached to the proximal end or the elongate body, wherein the proximal end or the elongate body is attached to a first surface of the baseplate configured to interface with the subject.

169. The implantable light conduit of claim 168, wherein the baseplate comprises an interface configured to attach to a microscope.
